(19) United States
(12) Patent Application Publication
(10) Pub. No.: US 2021/0330672 A1
(43) Pub. Date: Oct. 28, 2021

(54) USE OF BCL6 INHIBITORS FOR TREATING AUTOIMMUNE DISEASES

(71) Applicant: TEQLA THERAPEUTICS INC., Boston, MA (US)

(72) Inventors: Michael FRANKEN, Boston, MA (US); Stephen HALE, Boston, MA (US); Richard GARMAN, Boston, MA (US); Prabha IBRAHIM, Boston, MA (US)

(21) Appl. No.: 17/259,581

(22) PCT Filed: Jul. 12, 2019
(86) PCT No.: PCT/US19/41582

§ 371 Date: Jan. 12, 2021

Related U.S. Application Data
(60) Provisional application No. 62/697,489, filed on Jul. 13, 2018.

(51) Int. Cl. A61K 31/506; G01N 33/564

(52) U.S. Cl. CPC A61K 31/506; G01N 33/564

(57) ABSTRACT

Described herein include compositions and methods that can be useful for inhibiting activity of Tfh cells, B-cells, germinal centers, extrafollicular T and B-cells, and/or ectopic follicular structures; and/or in treating diseases such as autoimmune diseases.

### CROSS-REFERENCE

This application claims the benefit of U.S. Provisional Application No. 62/697,489, filed on Jul. 13, 2018, the contents of which are herein incorporated by reference in their entirety.

### BACKGROUND

CD4 T helper cells are critical for the immune response and are essential for helping B cells make high affinity antigen-specific antibody. Follicular helper T (Tfh) cells are a subset of CD4 T cells whose role is specifically to help B cells produce antibody, in part by promoting the germinal center reaction. Tfh cells are characterized by high expression of the transcription repressor BCL6 and secretion of the B cell stimulatory cytokine interleukin-21 (IL-21). It was shown that deregulation of development of Tfh cells can lead to autoimmune diseases and chronic inflammatory diseases.

### SUMMARY

Described herein are, inter alia, compositions and methods that are in some embodiments useful for inhibiting activity of Tfh cells, B-cells, germinal centers, extrafollicular T and B-cells, and/or ectopic follicular structures; and/or in treating diseases such as autoimmune diseases.

In one aspect, provided is a method that includes identifying a patient having ANCA associated vasculitis, antibody mediated transplant rejection, autoimmune hemolytic anemia, IgG4-related disease, chronic idiopathic demyelinating polyneuropathy (CIDP), graft-versus-host disease, or neuromyelitis optica and administering to the patient a compound of Formula (I).

In another aspect, provided is a method that includes identifying a patient as having one or more conditions selected from the group consisting of elevated numbers or activity of plasmablasts in blood or tissue, a plasmablast signature as determined by RNA expression or protein expression analyses, elevated numbers or activity of Tfh cells or Tfh-like cells in blood or tissue, elevated numbers or activity of germinal centers or extra follicular T and B-cells, presence of ectopic follicular structures or ectopic germinal centers or ectopic lymphoid aggregates in a tissue, and elevated levels of autoantibodies or immune complexes in blood or tissue; and administering to the patient a compound of Formula (I). In some embodiments, the patient is further diagnosed as having an autoimmune disease, for example an autoimmune disease such as disclosed herein.

In one aspect, provided is a method that includes identifying a patient as having one or more conditions selected from the group consisting of elevated numbers or activity of plasmablasts in blood or tissue, a plasmablast signature as determined by RNA expression or protein expression analyses, elevated numbers or activity of Tfh cells or Tfh-like cells in blood or tissue, elevated numbers or activity of germinal centers or extra follicular T and B-cells, presence of ectopic follicular structures or ectopic germinal centers or ectopic lymphoid aggregates in a tissue, and elevated levels of autoantibodies or immune complexes in blood or tissue; wherein the patient is further diagnosed as having an autoimmune disease (for example an autoimmune disease such as disclosed herein); and administering to the patient a compound of Formula (I).

In certain embodiments, the autoimmune disease is autoimmune disease is selected from the group consisting of lupus erythematosus, lupus nephritis, ANCA associated vasculitis, ankylosing spondylitis, antibody mediated transplant rejection, autoimmune hemolytic anemia, Chagas disease, chronic idiopathic demyelinating polyneuropathy (CIDP), chronic obstructive pulmonary disease, Crohn's Disease, dermatomyositis, diabetes mellitus type 1, endometriosis, Goodpasture syndrome, graft-versus-host disease, Graves' disease, Guillain-Barré syndrome (GBS), Hashimoto's disease, hidradenitis suppurativa, juvenile dermatomyositis, Kawasaki disease, IgA nephropathy, idiopathic thrombocytopenic purpura, IgG4-related disease, interstitial cystitis, mixed connective tissue disease, morphea, multiple sclerosis, secondary progressive multiple sclerosis, myasthenia gravis, narcolepsy, neuromyelitis optica, neuromyotonia, pemphigus vulgaris, pernicious anemia, psoriasis, psoriatic arthritis, polymyositis, primary biliary cirrhosis, relapsing polychondritis, rheumatoid arthritis, sarcoidosis, schizophrenia, scleroderma, Sjögren's syndrome, stiff person syndrome, temporal arteritis, ulcerative colitis, vasculitis, vitiligo, Wegener's granulomatosis, and any combination thereof. In certain embodiments, the autoimmune disease is autoimmune disease is selected from the group consisting of ANCA associated vasculitis, antibody mediated transplant rejection, autoimmune hemolytic anemia, IgG4-related disease, chronic idiopathic demyelinating polyneuropathy (CIDP), graft-versus-host disease, or neuromyelitis optica.

In one aspect, provided is a method that includes identifying a patient as having elevated numbers or activity of plasmablasts in blood or tissue and administering to the patient a compound of Formula (I). In some embodiments, the patient is further diagnosed as having an autoimmune disease, for example an autoimmune disease such as disclosed herein.

In one aspect, provided is a method that includes identifying a patient as having a plasmablast signature as determined by RNA expression or protein expression analyses; and administering to the patient a compound of Formula (I). In some embodiments, the patient is further diagnosed as having an autoimmune disease, for example an autoimmune disease such as disclosed herein.

In one aspect, provided is a method that includes identifying a patient as having elevated numbers or activity of Tfh cells or Tfh-like cells in blood or tissue; and administering to the patient a compound of Formula (I). In some embodiments, the patient is further diagnosed as having an autoimmune disease, for example an autoimmune disease such as disclosed herein.

In one aspect, provided is a method that includes identifying a patient as having elevated numbers or activity of germinal centers or extra follicular T and B-cells and administering to the patient a compound of Formula (I). In some embodiments, the patient is further diagnosed as having an autoimmune disease, for example an autoimmune disease such as disclosed herein.

In another aspect, provided is a method that includes identifying a patient as having the presence of ectopic follicular structures or ectopic germinal centers or ectopic lymphoid aggregates in a tissue, and administering to the patient a compound of Formula (I). In some embodiments, the patient is further diagnosed as having an autoimmune disease, for example an autoimmune disease such as disclosed herein.

In one aspect, provided is a method that includes identifying a patient as having elevated levels of autoantibodies or immune complexes in blood or tissue and administering to the patient a compound of Formula (I). In some embodiments, the patient is further diagnosed as having an autoimmune disease, for example an autoimmune disease such as disclosed herein.

As used herein “a compound of Formula (I)” means a compound having the following structure:

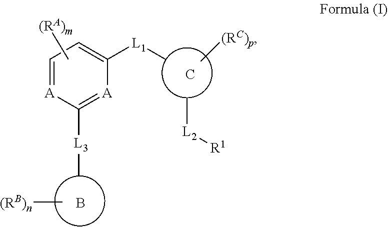

In some embodiments of Formula (1),
each A is independently N or CR;
ring B is heterocycloalkyl or heteroaryl;
ring C is 6-5 fused heterocycle, 6-6 fused heterocycle, or 5-6 fused heterocycle;
L₁ is —(CR²R³)ₓ—, —NR⁴—, —O—, —S—, —O—(CR²R³)ₓ—, —(CR²R³)ₓ—O—, —C(═O)—, —S(═O)—, —S(═O)₂—, —S(═O)₂NR⁴—, —NR⁴S(═O)₂—, —C(═O)O—, —OC(═O)—, —C(═O)NR⁴—, —NR⁴C(═O)—, —OC(═O)NR⁴—, —NR⁴C(═O)O—, or —NR⁴C(═O)NR⁴—;
L₂ and L₃ is independently absent, —O—(CR²R³)ₓ—, —(CR²R³)ₓ—O—, —NR⁴—(CR²R³)ₓ—, —(CR²R³)ₓ—NR⁴—, —C(═O)—, —S(═O)—, —S(═O)₂—, —S(═O)₂NR⁴—, —NR⁴S(═O)₂—, —C(═O)O—, —OC(═O)—, —C(═O)NR⁴—, —NR⁴C(═O)—, —OC(═O)NR⁴—, —NR⁴C(═O)O—, —NR⁴C(═O)NR⁴—, or —NR⁴—;
each R^{A}, R^{B}, R^{C}, R, R¹, R², and R³ is independently H, halogen, —CN, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted phenyl, or substituted or unsubstituted heteroaryl, —OR⁴, —N(R⁴)₂, —CH₂OR⁴, —C(═O)R⁴, —C(═O)OR⁴, —C(═O)N(R⁴)₂, —S(═O)R⁴, —S(═O)₂R⁴, or —NR⁴C(O)R⁴;

each R⁴ is independently H, D, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl;
each x is independently 1, 2, or 3;
m is 0, 1, 2, 3, or 4;
n is 0, 1, 2, 3, or 4; and
p is 0, 1, 2, 3, or 4.

In one aspect, described herein is a method of inhibiting Tfh activity in a subject in need thereof, comprising administering to the subject in need thereof a therapeutically effective amount of a BCL6 inhibitor, wherein the BCL6 inhibitor is a compound of Formula (I):

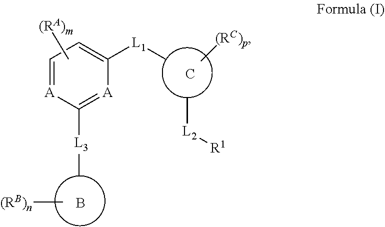

wherein,
each A is independently N or CR;
ring B is heterocycloalkyl or heteroaryl;
ring C is 6-5 fused heterocycle, 6-6 fused heterocycle, or 5-6 fused heterocycle;
L₁ is —(CR²R³)ₓ—, —NR⁴—, —O—, —S—, —O—(CR²R³)ₓ—, —(CR²R³)ₓ—O—, —C(═O)—, —S(═O)—, —S(═O)₂—, —S(═O)₂NR⁴—, —NR⁴S(═O)₂—, —C(═O)O—, —OC(═O)—, —C(═O)NR⁴—, —NR⁴C(═O)—, —OC(═O)NR⁴—, —NR⁴C(═O)O—, or —NR⁴C(═O)NR⁴—;
L₂ and L₃ is independently absent, —O—(CR²R³)ₓ—, —(CR²R³)ₓ—O—, —NR⁴—(CR²R³)ₓ—, —(CR²R³)ₓ—NR⁴—, —C(═O)—, —S(═O)—, —S(═O)₂—, —S(═O)₂NR⁴—, —NR⁴S(═O)₂—, —C(═O)O—, —OC(═O)—, —C(═O)NR⁴—, —NR⁴C(═O)—, —OC(═O)NR⁴—, —NR⁴C(═O)O—, —NR⁴C(═O)NR⁴—, or —NR⁴—;
each R^{A}, R^{B}, R^{C}, R, R¹, R², and R³ is independently H, halogen, —CN, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted phenyl, or substituted or unsubstituted heteroaryl, —OR⁴, —N(R⁴)₂, —CH₂OR⁴, —C(═O)R⁴, —C(═O)OR⁴, —C(═O)N(R⁴)₂, —S(═O)R⁴, —S(═O)₂R⁴, or —NR⁴C(═O)R⁴;
each R⁴ is independently H, D, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl; each x is independently 1, 2, or 3;
m is 0, 1, 2, 3, or 4;
n is 0, 1, 2, 3, or 4; and
p is 0, 1, 2, 3, or 4

In another aspect, described herein is a method of treating an autoimmune disease in a subject in need thereof, comprising administering to the subject in need thereof a therapeutically effective amount of a BCL6 inhibitor, wherein the BCL6 inhibitor is a compound of Formula (I):

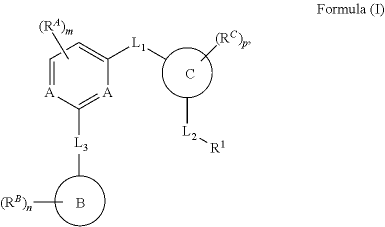

wherein,
each A is independently N or CR;
ring B is heterocycloalkyl or heteroaryl;
ring C is 6-5 fused heterocycle, 6-6 fused heterocycle, or 5-6 fused heterocycle;
L₁ is —(CR²R³)ₓ—, —NR⁴—, —O—, —S—, —O—(CR²R³)ₓ—, —(CR²R³)ₓ—O—, —C(═O)—, —S(═O)—, —S(═O)₂—, —S(═O)₂NR⁴—, —NR⁴S(═O)₂—, —C(═O)O—, —OC(═O)—, —C(═O)NR⁴—, —NR⁴C(═O)—, —OC(═O)NR⁴—, —NR⁴C(═O)O—, or —NR⁴C(═O)NR⁴—;
L₂ and L₃ is independently absent, —O—(CR²R³)ₓ—, —(CR²R³)ₓ—O—, —NR⁴—(CR²R³)ₓ—, —(CR²R³)ₓ—NR⁴—, —C(═O)—, —S(═O)—, —S(═O)₂—, —S(═O)₂NR⁴—, —NR⁴S(═O)₂—, —C(═O)O—, —OC(═O)—, —C(═O)NR⁴—, —NR⁴C(═O)—, —OC(═O)NR⁴—, —NR⁴C(═O)O—, —NR⁴C(═O)NR⁴—, or —NR⁴—;
each R^{A}, R^{B}, R^{C}, R, R¹, R², and R³ is independently H, halogen, —CN, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted phenyl, or substituted or unsubstituted heteroaryl, —OR⁴, —N(R⁴)₂, —CH₂OR⁴, —C(═O)R⁴, —C(═O)OR⁴, —C(═O)N(R⁴)₂, —S(═O)R⁴, —S(═O)₂R⁴, or —NR⁴C(═O)R⁴;
each R⁴ is independently H, D, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl; each x is independently 1, 2, or 3;
m is 0, 1, 2, 3, or 4;
n is 0, 1, 2, 3, or 4; and
p is 0, 1, 2, 3, or 4.

Any combination of the groups described above or below for the various variables is contemplated herein. Throughout the specification, groups and substituents thereof are chosen by one skilled in the field to provide stable moieties and compounds.

In another aspect, described herein is a pharmaceutical composition comprising a BCL6 inhibitor compound of Formula (I), or a pharmaceutically acceptable salt or solvate thereof, and at least one pharmaceutically acceptable excipient.

In some embodiments, a BCL6 inhibitor compound of Formula (I), or a pharmaceutically acceptable salt or solvate thereof, is formulated for administration to a mammal by intravenous administration, subcutaneous administration, oral administration, inhalation, nasal administration, dermal administration, or ophthalmic administration. In some embodiments, the BCL6 inhibitor compound of Formula (I), or a pharmaceutically acceptable salt or solvate thereof, is in the form of a tablet, a pill, a capsule, a liquid, a suspension, a gel, a dispersion, a solution, an emulsion, an ointment, or a lotion.

In another aspect, described herein is a method of inhibiting Tfh activity in a subject in need thereof, comprising administering to the subject in need thereof a therapeutically effective amount of a BCL6 inhibitor, wherein the BCL6 inhibitor is a compound of Formula (I), as a crystalline or amorphous form, or a pharmaceutically acceptable salt or solvate thereof, and a second therapeutic agent.

In another aspect, described herein is a method of treating an autoimmune disease in a subject in need thereof, comprising administering to the subject in need thereof a therapeutically effective amount of a BCL6 inhibitor, wherein the BCL6 inhibitor is a compound of Formula (I), or a pharmaceutically acceptable salt or solvate thereof, and a second therapeutic agent.

### DETAILED DESCRIPTION

CD4 T helper cells are critical for the immune response and are essential for helping B cells make high affinity antigen-specific antibody. Follicular helper T (Tfh) cells are a recently characterized subset of CD4 T cells whose role is specifically to help B cells produce antibody, in part by promoting the germinal center reaction. However, deregulated development of Tfh cells can lead to autoimmune disease. Tfh cells are localized to B cell follicles and thus express the chemokine receptor CXCR5. Tfh cells are also characterized by high expression of the transcription repressor BCL6 and secretion of the B cell stimulatory cytokine interleukin-21 (IL-21). It was shown that BCL6 is the master transcriptional regulator for Tfh cells—forced BCL6 expression can induce the Tfh phenotype in T cells, and Tfh cells cannot develop in the absence of BCL6.

CD4 T helper cells can differentiate into several different types of unique effector lineages (Th1, Th2, Th17, Treg, and Tfh). Each subset of cells mediates very different types of immune responses in part via the expression of different “signature” cytokines. Thus, Th1 cells produce Interferon-gamma and help to activate macrophages, Th2 cells produce IL-4 and IL-5 and promote allergic and anti-worm responses, Th17 cells produce IL-17 and IL-22 and promote inflammation and anti-bacterial immunity, Treg cells produce IL-10 and transforming growth factor (“TGF”)-beta and down-modulate the immune response, and Tfh cells produce IL-21 and help B cells form germinal centers and produce antibody. Development of the different subsets depends upon the cytokines the cells are exposed to during initial activation through the T cell receptor (“TCR”).

Further, the development of each specific CD4 subset requires a lineage-directing master transcription factor. Thus, Th1 cells require Tbet, Th2 cells require GATA3, Th17 cells require RORgammaT and Treg cells require FoxP3. The transcriptional repressor and B cell oncogene BCL6 is expressed at high levels in Tfh. It was shown that BCL6 is a master lineage-directing factor for Tfh cells. Forced BCL6 expression in CD4 T cells can strongly induce Tfh function in vivo, and BCL6 function is strictly required for the development of Tfh cells in vivo.

Tfh cells are characterized by high level of expression of the chemokine receptor CXCR5, which binds the chemokine CXCL13 that is expressed in B cell follicles. Thus, CXCL13 acting on CXCR5 promotes migration of Tfh cells to the B cell follicle. Tfh cells have an activated effector T cell phenotype and express ICOS, OX40, CD4OL, CD44 and BTLA, and are negative for CCR7 and CD62. Tfh cells have been characterized as expressing PD1 and CD200; however, high levels of CXCR5 and decreased levels of SLAM may be the most specific set of markers for Tfh cells. In addition, Tfh cells have been found to be characterized by a BCL6-dependent downregulation of P-selectin glycoprotein ligand 1 (PSGL1, a CCL19- and CCL21-binding protein), indicating that, like CXCR5 and PD1 upregulation, modulation of PSGL1 expression is part of the Tfh phenotype.

There are two major stages of Tfh function. First, “pregerminal center” Tfh cells interact with antigen-activated B cells and promote the major phases of the initial B cell response: B cell clonal expansion, antibody isotype switch, plasma cell differentiation, and the induction of germinal centers. Second, Tfh cells regulate the fate of B cells and the antibody response by interacting with B cells in the germinal center. Thus, Tfh cells are critical for memory B cell and plasma cell development. The key cytokine produced by Tfh cells is IL-21, which is a factor that potently promotes B cell activation and antibody secretion.

Tfh cells have been shown to be critical for the proper production of antibody, which is a central and vital component of adaptive immunity. At the same time, the over-production of Tfh cells can lead to autoimmunity as Tfh cells help B cells to produce self-reactive antibodies. This has been most clearly observed with sanroque mice, where a mutation in the roquin gene leads to increased ICOS expression, uncontrolled Tfh development and lupus-like autoimmune disease.

High-level expression of BCL6 in germinal center T cells had been reported, but germinal center T cells were poorly understood until the Tfh subset became characterized. BCL6 was shown to be required for Tfh cell function. In particular, it was shown that infecting wild-type CD4 T cells with BCL6 retrovirus (RV) greatly augmented the ability of CD4 T cells to become Tfh cells in vivo. This suggests that BCL6 can actively promote development of the Tfh phenotype.

Based on importance of BCL6 in transcriptional regulation of Tfh cells, the compounds which alter BCL6 activity are considered to be useful in treating or preventing autoimmune diseases. Accordingly, in one aspect, described herein is a method of inhibiting Tfh activity in a subject in need thereof, comprising administering to the subject in need thereof a therapeutically effective amount of a BCL6 inhibitor. In another aspect, described herein is a method of treating an autoimmune disease in a subject in need thereof, comprising administering to the subject in need thereof a therapeutically effective amount of a BCL6 inhibitor.

### Compounds

In one aspect, described herein is a BCL6 inhibitor compound, having the structure of Formula (I), or a pharmaceutically acceptable salt or solvate thereof:

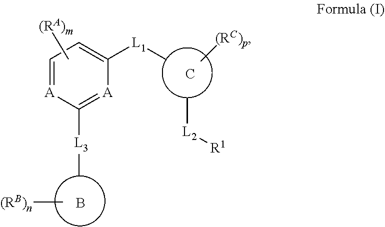

wherein,
each A is independently N or CR;
ring B is heterocycloalkyl or heteroaryl;
ring C is 6-5 fused heterocycle, 6-6 fused heterocycle, or 5-6 fused heterocycle;
L₁ is —(CR²R³)ₓ—, —NR⁴—, —O—, —S—, —O—(CR²R³)ₓ—, —(CR²R³)ₓ—O—, —C(═O)—, —S(═O)—, —S(═O)₂—, —S(═O)₂NR⁴—, —NR⁴S(═O)₂—, —C(═O)O—, —OC(═O)—, —C(═O)NR⁴—, —NR⁴C(═O)—, —OC(═O)NR⁴—, —NR⁴C(═O)O—, or —NR⁴C(═O)NR⁴—;
L₂ and L₃ is independently absent, —O—(CR²R³)ₓ—, —(CR²R³)ₓ—O—, —NR⁴—(CR²R³)ₓ—, —(CR²R³)ₓ—NR⁴—, —C(═O)—, —S(═O)—, —S(═O)₂—, —S(═O)₂NR⁴—, —NR⁴S(═O)₂—, —C(═O)O—, —OC(═O)—, —C(═O)NR⁴—, —NR⁴C(═O)—, —OC(═O)NR⁴—, —NR⁴C(═O)O—, —NR⁴C(═O)NR⁴—, or —NR⁴—;
each R^{A}, R^{B}, R^{C}, R, R¹, R², and R³ is independently H, halogen, —CN, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted phenyl, or substituted or unsubstituted heteroaryl, —OR⁴, —N(R⁴)₂, —CH₂OR⁴, —C(═O)R⁴, —C(═O)OR⁴, —C(═O)N(R⁴)₂, —S(═O)R⁴, —S(═O)₂R⁴, or —NR⁴C(═O)R⁴;
each R⁴ is independently H, D, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl;
each x is independently 1, 2, or 3;
m is 0, 1, 2, 3, or 4;
n is 0, 1, 2, 3, or 4; and
p is 0, 1, 2, 3, or 4.

In some embodiments, each A is N. In some embodiments, each A is CR. In some embodiments, each A is independently N or CR, provided that at least one A is N.

In some embodiments, each R^{A} is independently halogen, —CN, substituted or unsubstituted C₁-C₆alkyl, substituted or unsubstituted C₁-C₆fluoroalkyl, or substituted or unsubstituted C₁-C₆heteroalkyl.

In some embodiments, each R^{A} is independently halogen, —CN, or substituted or unsubstituted C₁-C₆heteroalkyl. In some embodiments, each R^{A} is independently halogen or substituted or unsubstituted C₁-C₆heteroalkyl.

In some embodiments, R^{A} is halogen. In some embodiments, R^{A} is F, Cl, or Br. In some embodiments, R^{A} is F. In some embodiments, R^{A} is Cl. In some embodiments, R^{A} is Br.

In some embodiments, L₁ is —(CR²R³)ₓ—, —NR⁴—, —O—, —C(═O)—, —S(═O)—, or —S(═O)₂—, wherein x is 1 or 2. In some embodiments, L₁ is —NR⁴—, —O—, —C(═O)—, —S(═O)—, or —S(═O)₂—. In some embodiments, L₁ is —NR⁴—, —O—, or —C(═O)—. In some embodiments, L₁ is —NR⁴—. In some embodiments, L₁ is —O—. In some embodiments, L₁ is —C(═O)—.

In some embodiments, ring C is selected from the group consisting of:

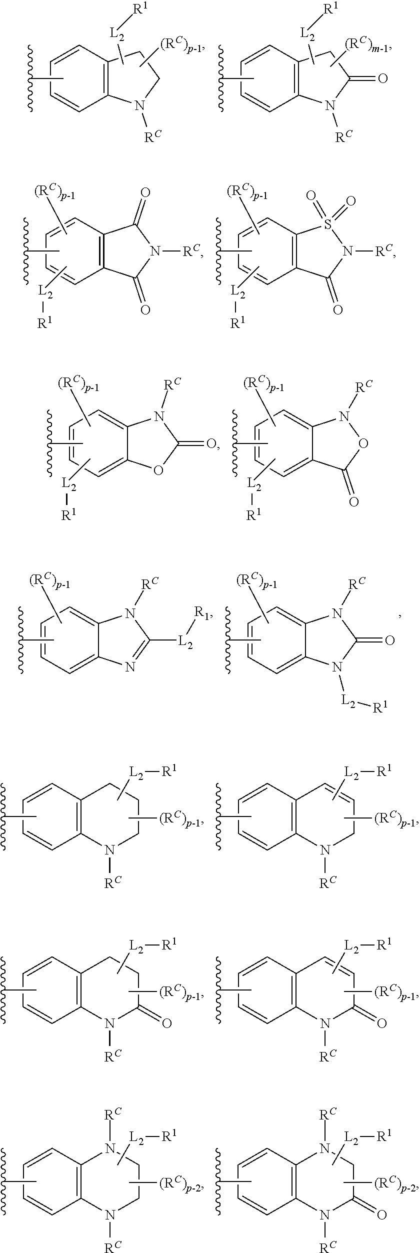

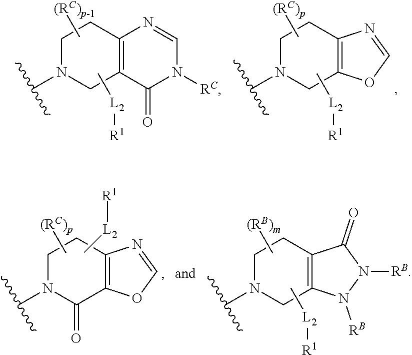

In some embodiments, ring C is

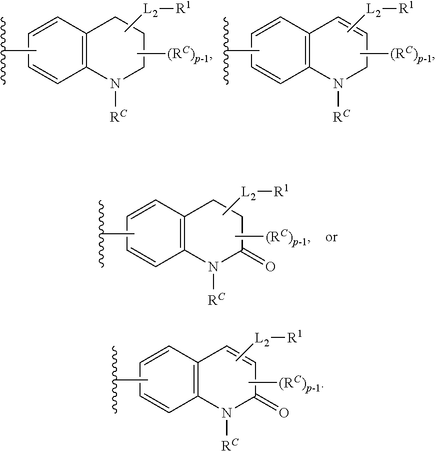

In some embodiments, ring C is

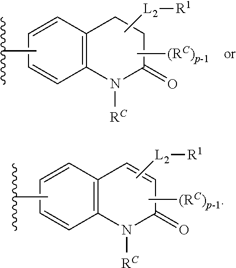

In some embodiments, ring C is

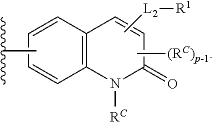

In some embodiments, R^{C} is substituted or unsubstituted C₁-C₆alkyl, substituted or unsubstituted C₁-C₆heteroalkyl, or —OR⁴. In some embodiments, R^{C} is substituted or unsubstituted C₁-C₆alkyl. In some embodiments, R^{C} is substituted or unsubstituted C₁-C₆heteroalkyl. In some embodiments, R^{C} is —OR⁴.

In some embodiments, L² is —O—(CR²R³)ₓ—, —(CR²R³)ₓ—O—, —NR⁴—(CR²R³)ₓ—, or —(CR²R³)ₓ—NR⁴—, and x is 1 or 2. In some embodiments, L² is —O—(CR²R³)ₓ— or —(CR²R³)ₓ—O—, and x is 1 or 2. In some embodiments, L² is —O—(CR²R³)ₓ—. In some embodiments, L² is —O—(CR²R³)ₓ—, and x is 1 or 2. In some embodiments, L² is —(CR²R³)ₓ—O—. In some embodiments, L² is —(CR²R³)ₓ—O—, and x is 1 or 2.

In some embodiments, R¹ is —OR⁴, —N(R⁴)₂, —CH₂OR⁴, —C(═O)R⁴, —C(═O)OR⁴, —C(═O)N(R⁴)₂, —S(═O)R⁴, —S(═O)₂R⁴, or —NR⁴C(═O)R⁴. In some embodiments, R¹ is —C(═O)R⁴, —C(═O)OR⁴, —C(═O)N(R⁴)₂, —S(═O)R⁴, —S(═O)₂R⁴, or —NR⁴C(═O)R⁴. In some embodiments, R¹ is —C(═O)R⁴, —C(═O)OR⁴, —C(═O)N(R⁴)₂, —S(═O)₂R⁴, or —NR⁴C(═O)R⁴. In some embodiments, R¹ is —C(═O)R⁴. In some embodiments, R¹ is —C(═O)OR⁴. In some embodiments, R¹ is —C(═O)N(R⁴)₂. In some embodiments, R¹ is —S(═O)₂R⁴. In some embodiments, R¹ is —NR⁴C(═O)R⁴.

In some embodiments, L₃ is absent or —C(═O)—. In some embodiments, L₃ is absent. In some embodiments, L₃ is —C(═O)—.

In some embodiments, ring B is heterocycloalkyl. In some embodiments, ring B is

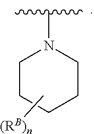

In some embodiments, ring B is

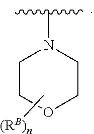

In some embodiments, ring B is

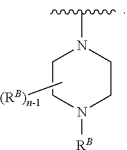

In some embodiments, R^{B} is independently substituted or unsubstituted C₁-C₆alkyl, substituted or unsubstituted C₁-C₆fluoroalkyl, substituted or unsubstituted C₁-C₆heteroalkyl, —C(═O)R⁴, —C(═O)OR⁴, —C(═O)N(R⁴)₂, —S(═O)R⁴, —S(═O)₂R⁴, or —NR⁴C(═O)R⁴. In some embodiments, R^{B} is independently substituted or unsubstituted C₁-C₆alkyl, substituted or unsubstituted C₁-C₆heteroalkyl, —C(═O)R⁴, —C(═O)OR⁴, —C(═O)N(R⁴)₂, —S(═O)₂R⁴, or —NR⁴C(═O)R⁴. In some embodiments, R^{B} is independently substituted or unsubstituted C₁-C₆alkyl, substituted or unsubstituted C₁-C₆heteroalkyl, —C(═O)R⁴, —C(═O)N(R⁴)₂, or —NR⁴C(═O)R⁴. In some embodiments, R^{B} is independently substituted or unsubstituted C₁-C₆alkyl, —C(═O)R⁴, —C(═O)N(R⁴)₂, or —NR⁴C(═O)R⁴. In some embodiments, R^{B} is substituted or unsubstituted C₁-C₆alkyl or —C(═O)N(R⁴)₂.

In some embodiments, n is 1 or 2. In some embodiments, n is 1. In some embodiments, n is 2.

In some embodiments, ring C is

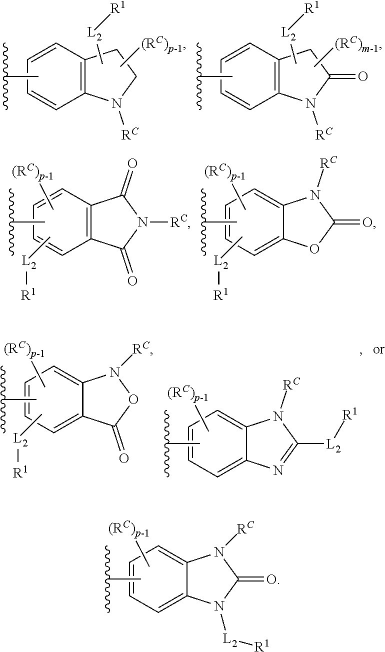

In some embodiments, ring C is

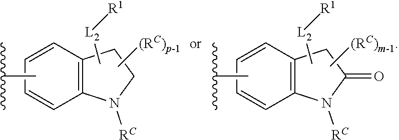

In some embodiments, ring C is

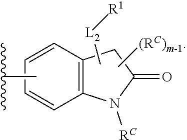

In some embodiments, ring C is

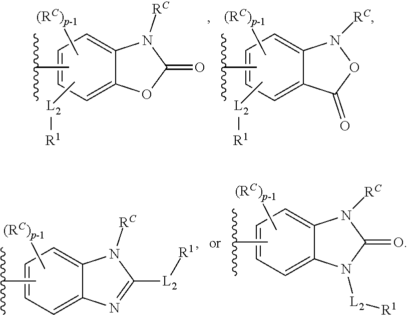

In some embodiments, L₂ is absent or —C(═O)—. In some embodiments, L₂ is absent. In some embodiments, L₂ is —C(═O)—.

In some embodiments, R¹ and R^{C} are each independently H, halogen, —CN, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted phenyl, or substituted or unsubstituted heteroaryl, —OR⁴, —N(R⁴)₂, —CH₂OR⁴,

In some embodiments, L₃ is —NR⁴(CR₂R³)ₓ—, —(CR₂R³)ₓ—NR⁴—, —S(═O)₂NR⁴—, or —NR⁴S(═O)₂—. In some embodiments, L₃ is —NR⁴—(CR²R³)ₓ— or —(CR²R³)ₓ—NR⁴—. In some embodiments, L₃ is —NR⁴—(CR²R³)ₓ—. In some embodiments, L₃ is —(CR²R³)ₓ—NR⁴—.

In some embodiments, ring B is heteroaryl. In some embodiments, ring B is

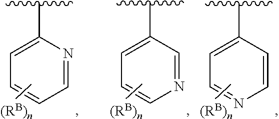

In some embodiments, ring B is

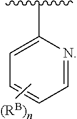

In some embodiments, ring B is

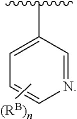

In some embodiments, ring B is

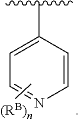

Any combination of the groups described above or below for the various variables is contemplated herein. Throughout the specification, groups and substituents thereof are chosen by one skilled in the field to provide stable moieties and compounds.

In some embodiments, a compound of Formula (I) is a compound selected from Table 1.

TABLE 1

| Compound # | Structure | Compound Name |
|---|---|---|
| Compound 1 | 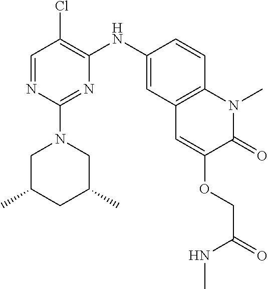 [SMILES: CNC(=O)COc1cc2cc(Nc3nc(N4C[C@H](C)C[C@H](C)C4)ncc3Cl)ccc2n(C)c1=O] | 2-((6-((5-chloro-2-((3R,5S)-3,5- dimethylpiperidin-1-yl)pyrimidin-4- yl)amino)-1-methyl-2-oxo-1,2- dihydroquinolin-3-yl)oxy)-N- methylacetamide |
| Compound 2 | 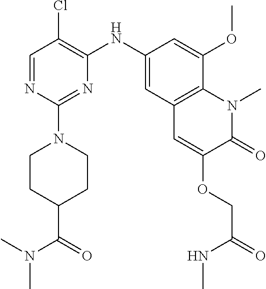 [SMILES: CNC(=O)COc1cc2cc(Nc3nc(N4CCC(C(=O)N(C)C)CC4)ncc3Cl)cc(OC)c2n(C)c1=O] | 1-(5-chloro-4-((8-methoxy-1- methyl-3-(2-(methylamino)-2- oxoethoxy)-2-oxo-1,2- dihydroquinolin-6- yl)amino)pyrimidin-2-yl)-N,N- dimethylpiperidine-4-carboxamide |
| Compound 3 | 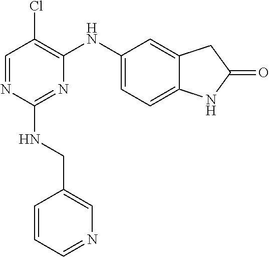 [SMILES: O=C1Cc2cc(Nc3nc(NCc4cccnc4)ncc3Cl)ccc2N1] | 5-((5-chloro-2-((pyridin-3- ylmethyl)amino)pyrimidin-4- yl)amino)indolin-2-one |

In one aspect, provided herein is a pharmaceutically acceptable salt or solvate thereof of a compound described in Table 1.

### Further Forms of Compounds

In one aspect, a compound of Formula (I) possesses one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In one aspect, stereoisomers are obtained by stereoselective synthesis.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

“Pharmaceutically acceptable” as used herein, refers a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound of Formula (I) with acids. Pharmaceutically acceptable salts are also obtained by reacting a compound of Formula (I) with a base to form a salt.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent, such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Synthesis of Compounds

Compounds described herein are synthesized using standard synthetic techniques or using methods known in the art in combination with methods described herein. In some embodiments, compounds described herein are purchased from commercial sources.

Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6th Edition, John Wiley and Sons, Inc. Alternative reaction conditions for the synthetic transformations described herein may be employed such as variation of solvent, reaction temperature, reaction time, as well as different chemical reagents and other reaction conditions. The starting materials are available from commercial sources or are readily prepared.

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3 527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

In the reactions described, it may be necessary to protect reactive functional groups, for example hydroxy, amino, imino, thio or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure).

In some embodiments, compounds are synthesized as described in the Examples section. In some embodiments, compounds are synthesized as described in Kamada, Y. et al. J. Med. Chem. 2017, 60, 4358-4368. In some embodiments, compounds are synthesized as described in Kerres, N. et al. Cell Reports 2017, 20, 2860-2875.

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

The term “oxo” refers to the ═O substituent.

The term “thioxo” refers to the ═S substituent.

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

The term “alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

The term “alkylamino” refers to a radical of the formula —NHR or —NRR where each R is, independently, an alkyl radical as defined above. Unless stated otherwise specifically in the specification, an alkylamino group may be optionally substituted as described below. The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃ —C≡CCH₂CH₃, —CH₂C≡CH.

The term “aromatic” refers to a planar ring having a delocalized π-electron system containing 4n+2 π electrons, where n is an integer. Aromatics can be optionally substituted. The term “aromatic” includes both aryl groups (e.g., phenyl, naphthalenyl) and heteroaryl groups (e.g., pyridinyl, quinolinyl).

The terms “carbocyclic” or “carbocycle” refer to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycle includes cycloalkyl and aryl.

The term “aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group is partially reduced to form a cycloalkyl group defined herein. In some embodiments, an aryl group is fully reduced to form a cycloalkyl group defined herein.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopropyl, cyclobutyl, cyclopentyl or cyclohexyl. In some embodiments, the monocyclic cycloalkyl is cyclopentyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “bridged” refers to any ring structure with two or more rings that contains a bridge connecting two bridgehead atoms. The bridgehead atoms are defined as atoms that are the part of the skeletal framework of the molecule and which are bonded to three or more other skeletal atoms. In some embodiments, the bridgehead atoms are C, N, or P. In some embodiments, the bridge is a single atom or a chain of atoms that connects two bridgehead atoms. In some embodiments, the bridge is a valence bond that connects two bridgehead atoms. In some embodiments, the bridged ring system is cycloalkyl. In some embodiments, the bridged ring system is heterocycloalkyl.

The term “fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with one or more N, S, and O atoms. The non-limiting examples of fused heterocyclyl or heteroaryl ring structures include 6-5 fused heterocycle, 6-6 fused heterocycle, 5-6 fused heterocycle, 5-5 fused heterocycle, 7-5 fused heterocycle, and 5-7 fused heterocycle.

The term “halo” or “halogen” refers to bromo, chloro, fluoro or iodo.

The term “haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

The term “haloalkoxy” refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

The term “fluoroalkyl” refers to an alkyl in which one or more hydrogen atoms are replaced by a fluorine atom. In one aspect, a fluoralkyl is a C₁-C₆fluoroalkyl. In some embodiments, a fluoroalkyl is selected from trifluoromethyl, difluoromethyl, fluoromethyl, 2,2,2-trifluoroethyl, 1-fluoromethyl-2-fluoroethyl, and the like.

The term “heteroalkyl” refers to an alkyl group in which one or more skeletal atoms of the alkyl are selected from an atom other than carbon, e.g., oxygen, nitrogen (e.g. —NH—, —N(alkyl)-, or —N(aryl)-), sulfur (e.g. —S—, —S(═O)—, or —S(═O)₂—), or combinations thereof. In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₆heteroalkyl. Representative heteroalkyl groups include, but are not limited to —OCH₂OMe, —OCH₂CH₂OH, —OCH₂CH₂OMe, or —OCH₂CH₂OCH₂CH₂NH₂.

The term “heteroalkylene” refers to an alkyl radical as described above where one or more carbon atoms of the alkyl is replaced with a O, N or S atom. “Heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkylene groups include, but are not limited to —OCH₂CH₂O—, —OCH₂CH₂OCH₂CH₂O—, or —OCH₂CH₂OCH₂CH₂OCH₂CH₂O—.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heterocycle” or “heterocyclic” refers to heteroaromatic rings (also known as heteroaryls) and heterocycloalkyl rings (also known as heteroalicyclic groups) that includes at least one heteroatom selected from nitrogen, oxygen and sulfur, wherein each heterocyclic group has from 3 to 12 atoms in its ring system, and with the proviso that any ring does not contain two adjacent O or S atoms. In some embodiments, heterocycles are monocyclic, bicyclic, polycyclic, spirocyclic or bridged compounds. Non-aromatic heterocyclic groups (also known as heterocycloalkyls) include rings having 3 to 12 atoms in its ring system and aromatic heterocyclic groups include rings having 5 to 12 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The term “heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, a bicyclic heteroaryl is a C₆-C₉heteroaryl. In some embodiments, a heteroaryl group is partially reduced to form a heterocycloalkyl group defined herein. In some embodiments, a heteroaryl group is fully reduced to form a heterocycloalkyl group defined herein.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

The term “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

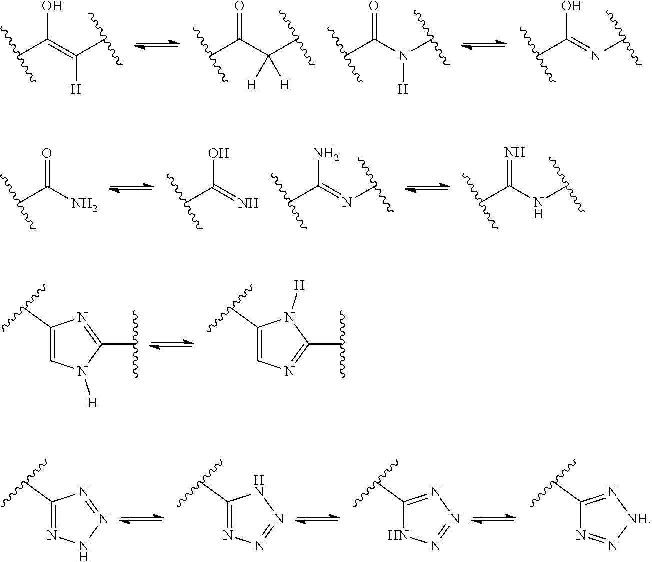

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound of Formula (I) and a co-agent, are both administered to a patient simultaneously in the form of a single entity or dosage. The term “non-fixed combination” means that the active ingredients, e.g. a compound of Formula (I) and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “kit” and “article of manufacture” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Administration and Pharmaceutical Composition

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

A pharmaceutical composition, as used herein, refers to a mixture of a compound of Formula (I) with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism.

Pharmaceutical formulations described herein are administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the compounds of Formula (I) are administered orally.

In some embodiments, the compounds of Formula (I) are administered topically. In such embodiments, the compound of Formula (I) is formulated into a variety of topically administrable compositions, such as solutions, suspensions, lotions, gels, pastes, shampoos, scrubs, rubs, smears, medicated sticks, medicated bandages, balms, creams or ointments. In one aspect, the compounds of Formula (I) are administered topically to the skin.

In another aspect, the compounds of Formula (I) are administered by inhalation.

In another aspect, the compounds of Formula (I) are formulated for intranasal administration. Such formulations include nasal sprays, nasal mists, and the like.

In another aspect, the compounds of Formula (I) are formulated as eye drops.

In any of the aforementioned aspects are further embodiments in which the effective amount of the compound of Formula (I) is: (a) systemically administered to the mammal; and/or (b) administered orally to the mammal; and/or (c) intravenously administered to the mammal; and/or (d) administered by inhalation to the mammal; and/or (e) administered by nasal administration to the mammal; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the aforementioned aspects are further embodiments comprising single administrations of the effective amount of the compound of Formula (I), including further embodiments in which (i) the compound is administered once; (ii) the compound is administered to the mammal multiple times over the span of one day; (iii) continually; or (iv) continuously.

In any of the aforementioned aspects are further embodiments comprising multiple administrations of the effective amount of the compound of Formula (I), including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound of Formula (I) is temporarily suspended or the dose of the compound being administered is temporarily reduced; at the end of the drug holiday, dosing of the compound is resumed. In one embodiment, the length of the drug holiday varies from 2 days to 1 year.

In certain embodiments, the compound of Formula (I) is administered in a local rather than systemic manner.

In some embodiments, the compound of Formula (I) is administered topically. In some embodiments, the compound of Formula (I) is administered systemically.

In some embodiments, the pharmaceutical formulation is in the form of a tablet. In other embodiments, pharmaceutical formulations of the compounds of Formula (I) are in the form of a capsule.

In one aspect, liquid formulation dosage forms for oral administration are in the form of aqueous suspensions or solutions selected from the group including, but not limited to, aqueous oral dispersions, emulsions, solutions, elixirs, gels, and syrups.

For administration by inhalation, a compound of Formula (I) is formulated for use as an aerosol, a mist or a powder.

For buccal or sublingual administration, the compositions may take the form of tablets, lozenges, or gels formulated in a conventional manner.

In some embodiments, compounds of Formula (I) are prepared as transdermal dosage forms.

In some embodiments, the compound of Formula (I) is be administered topically and can be formulated into a variety of topically administrable compositions, such as solutions, suspensions, lotions, gels, pastes, medicated sticks, balms, creams or ointments.

In some embodiments, the compounds of Formula (I) are formulated in rectal compositions such as enemas, rectal gels, rectal foams, rectal aerosols, suppositories, jelly suppositories, or retention enemas.

### Methods of Dosing and Treatment Regimens

In one embodiment, the compounds of Formula (I) are used in the preparation of medicaments for the treatment of diseases or conditions described herein. In addition, a method for treating any of the diseases or conditions described herein in a subject in need of such treatment, involves administration of pharmaceutical compositions that include at least one compound of Formula (I) or a pharmaceutically acceptable salt, active metabolite, prodrug, or solvate thereof, in therapeutically effective amounts to the subject.

In certain embodiments, the compositions containing the compound of Formula (I) are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions containing the compounds of Formula (I) are administered to a patient susceptible to or otherwise at risk of a particular disease, disorder or condition.

In certain embodiments, the dose of drug being administered may be temporarily reduced or temporarily suspended for a certain length of time (i.e., a “drug holiday”).

Doses employed for adult human treatment are typically in the range of 0.01 mg-5000 mg per day or from about 1 mg to about 1000 mg per day. In one embodiment, the desired dose is conveniently presented in a single dose or in divided doses.

In some embodiments, the pharmaceutical compositions disclosed herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In some embodiments, one or more pharmaceutical compositions are administered simultaneously, sequentially, or at an interval period of time. In some embodiments, one or more pharmaceutical compositions are administered simultaneously. In some cases, one or more pharmaceutical compositions are administered sequentially. In additional cases, one or more pharmaceutical compositions are administered at an interval period of time (e.g., the first administration of a first pharmaceutical composition is on day one followed by an interval of at least 1, 2, 3, 4, 5, or more days prior to the administration of at least a second pharmaceutical composition).

In some embodiments, two or more different pharmaceutical compositions are co-administered. In some instances, the two or more different pharmaceutical compositions are co-administered simultaneously. In some cases, the two or more different pharmaceutical compositions are co-administered sequentially without a gap of time between administrations. In other cases, the two or more different pharmaceutical compositions are co-administered sequentially with a gap of about 0.5 hour, 1 hour, 2 hour, 3 hour, 12 hours, 1 day, 2 days, or more between administrations.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of a given agent that corresponds to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Methods of Treatment

One embodiment provides a compound of Formula (I), or a pharmaceutically acceptable salt or solvate thereof, for use in a method of treatment of the human or animal body.

One embodiment provides a compound of Formula (I), or a pharmaceutically acceptable salt or solvate thereof, for use in a method of treatment of an autoimmune disease.

One embodiment provides a use of a compound of Formula (I), or a pharmaceutically acceptable salt or solvate thereof, in the manufacture of a medicament for the treatment of an autoimmune disease.

In some embodiments, described herein is a method of treating an autoimmune disease in a patient in need thereof comprising administering to the patient a compound of Formula (I), or a pharmaceutically acceptable salt or solvate thereof. In some embodiments, described herein is a method of treating an autoimmune disease in a patient in need thereof comprising administering to the patient a compound disclosed in Table 1, or a pharmaceutically acceptable salt or solvate thereof. In some embodiments, also described herein is a method of treating an autoimmune disease in a patient in need thereof comprising administering to the patient a pharmaceutical composition comprising a compound of Formula (I), or a pharmaceutically acceptable salt or solvate thereof, and a pharmaceutically acceptable excipient. In some embodiments, also described herein is a method of treating an autoimmune disease in a patient in need thereof comprising administering to the patient a pharmaceutical composition comprising a compound disclosed in Table 1, or a pharmaceutically acceptable salt or solvate thereof, and a pharmaceutically acceptable excipient. One embodiment provides a compound of Formula (I), or a pharmaceutically acceptable salt or solvate thereof, for use in a method of treatment of an autoimmune disease.

In some embodiments of any of the aspects and embodiments contemplated herein, the autoimmune disease may be selected from the group consisting of lupus erythematosus, lupus nephritis, ANCA associated vasculitis, ankylosing spondylitis, antibody mediated transplant rejection, autoimmune hemolytic anemia, Chagas disease, chronic idiopathic demyelinating polyneuropathy (CIDP), chronic obstructive pulmonary disease, Crohn's Disease, dermatomyositis, diabetes mellitus type 1, endometriosis, Goodpasture syndrome, graft-versus-host disease, Graves' disease, Guillain-Barré syndrome (GBS), Hashimoto's disease, hidradenitis suppurativa, juvenile dermatomyositis, Kawasaki disease, IgA nephropathy, idiopathic thrombocytopenic purpura, IgG4-related disease, interstitial cystitis, mixed connective tissue disease, morphea, multiple sclerosis, secondary progressive multiple sclerosis, myasthenia gravis, narcolepsy, neuromyelitis optica, neuromyotonia, pemphigus vulgaris, pernicious anemia, psoriasis, psoriatic arthritis, polymyositis, primary biliary cirrhosis, relapsing polychondritis, rheumatoid arthritis, sarcoidosis, schizophrenia, scleroderma, Sjögren's syndrome, stiff person syndrome, temporal arteritis, ulcerative colitis, vasculitis, vitiligo, Wegener's granulomatosis, and any combination thereof. In some embodiments, the autoimmune disease is lupus erythematosus. In some embodiments, the lupus erythematosus is lupus nephritis. In some embodiments, the autoimmune disease is IgG4-related disease. In some embodiments, the autoimmune disease is Sjögren's syndrome. In some embodiments, the autoimmune disease is rheumatoid arthritis. In some embodiments, the autoimmune disease is juvenile dermatomyositis. In some embodiments, the autoimmune disease is multiple sclerosis.

One embodiment provides a use of a compound of Formula (I), or a pharmaceutically acceptable salt or solvate thereof, in the manufacture of a medicament for the treatment of and autoimmune disease. In some embodiments, the autoimmune disease is lupus erythematosus. In some embodiments, the lupus erythematosus is lupus nephritis. In some embodiments, the autoimmune disease is IgG4-related disease. In some embodiments, the autoimmune disease is Sjögren's syndrome. In some embodiments, the autoimmune disease is rheumatoid arthritis. In some embodiments, the autoimmune disease is juvenile dermatomyositis. In some embodiments, the autoimmune disease is multiple sclerosis.

One embodiment provides a compound of Formula (I), or a pharmaceutically acceptable salt or solvate thereof, for use in a method of treatment of an autoimmune disease. In some embodiments, the compound of Formula (I) inhibits non-specific stimulation of B cells. In some embodiments, the compound of Formula (I) ameliorates autoimmune disease symptoms in the subject. In some embodiments, the compound of Formula (I) reduces secretion of auto-antibodies by B cells in the subject in vitro. In some embodiments, the compound of Formula (I) reduces secretion of auto-antibodies by B cells in the subject in vivo.

In some embodiments, the method further comprises detecting a first level of one or more auto-antibodies and activated Tfh cells in a first blood sample derived from the subject before administering the compound of Formula (I).

In some embodiments, the first level of one or more of auto-antibodies and activated Tfh cells in the first blood sample derived from the subject is increased compare to a control before administering the compound of Formula (I).

In certain embodiments, one or more of the following patient criteria/characteristics may be used to identify patients for use in the methods provided herein: (1) evaluating numbers or activity of plasmablasts in blood or tissue, (2) determining whether the patient has a plasmablast signature as determined by RNA expression or protein expression analyses, (3) determining numbers or activity of Tfh cells or Tfh-like cells in blood or tissue, (4) determining numbers or activity of germinal centers or extra follicular T and B-cells, (5) identifying the presence of ectopic follicular structures or ectopic germinal centers or ectopic lymphoid aggregates in a tissue, and (6) determining levels of autoantibodies or immune complexes in blood or tissue. Evaluating numbers or activity of plasmablasts in blood or tissue and determining whether the patient has a plasmablast signature as determined by RNA expression or protein expression analyses, can be performed, for example, as described in Wallace et al. 2015. Ann. Rheum. Dis. 74:190-195 and Banchereau et al. 2016. Cell 165:551-565. Determining numbers or activity of Tfh cells or Tfh-like cells in blood or tissue can be performed, for example, as reviewed in Craft. 2012. Nat. Rev. Rheum. 8:337-347. Crotty. 2014. Immunity 41:529-542 provides exemplary ways numbers or activity of germinal centers or extra follicular T and B-cells can be determined. Identifying the presence of ectopic follicular structures or ectopic germinal centers or ectopic lymphoid aggregates in a tissue can be achieved, for example as described in Kim et al. 2018. J. Immunol. 201:1359-1372. Determining levels of autoantibodies or immune complexes in blood or tissue can be performed, for example, as reviewed in Jones and Jones. 2015. Immunology 147:141-151 and Corsieri et al. 2016. Front. Immunol. 7:1-6.

In some embodiments, the method further comprises detecting a second level of one or more of auto-antibodies and activated Tfh cells in a second blood sample derived from the subject after administering the compound of Formula (I). In some embodiments, the second level of one or more of auto-antibodies and activated Tfh cells in the second blood sample derived from the subject is decreased compare to the first level after administering the compound of Formula (I). In some embodiments, the auto-antibody comprises plasmablasts antibodies. In some embodiments, the auto-antibody comprises IL-21 antibodies. In some embodiments, the auto-antibody comprises CXCL13 antibodies. In some embodiments, the auto-antibody comprises IgG and IgM antibodies. In some embodiments, the compound of Formula (I) inhibits activation, differentiation or re-activation of Tfh cells.

In some embodiments, the first level and second level of one or more of auto-antibodies and activated Tfh cells is determined in peripheral blood. In some embodiments, the first level and second level of one or more of auto-antibodies and activated Tfh cells is determined in serum or plasma.

### Combination Treatments

In certain instances, it is appropriate to administer at least one compound of Formula (I) in combination with another therapeutic agent.

In one specific embodiment, a compound of Formula (I) is co-administered with a second therapeutic agent, wherein the compound of Formula (I) and the second therapeutic agent modulate different aspects of the disease, disorder or condition being treated, thereby providing a greater overall benefit than administration of either therapeutic agent alone.

In some embodiments, the disease, disorder or condition being treated is an autoimmune disease. In some embodiments, the second therapeutic agent is selected from the group consisting of actarit, allocupreide sodium, bucillamine, clobuzarit, cuproxoline, diacerein, glucosamine, kebuzone, lobenzarit, melittin, myoral, methotrexate, leflunomide, cyclosporine, sulfasalazine, azathioprine, penicillamine, cyclophosphamide, and minocycline.

In some embodiments, the second therapeutic agent is selected from the group consisting of a non steroidal anti-inflammatory drug (NSAID) such as acetylsalicylic, ibuprofen, diclofenac, tenoxicam, naproxen or cyclooxygenase-2 inhibitors, e.g. celecoxib, rofecoxib, and valdecoxib.

In some embodiments, the second therapeutic agent is selected from the group consisting of an anti-inflammatory steroidal drug such as 21-acetoxypregnenolone, alclometasone, algestone, ancinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, maziprednone, medrysone, meprednisone, methyiprednisolone, mometasone furoate, paramethasone, prednicarbate, prednisolone, prednisolone 25-diethylamino acetate, prednisolone sodium phosphate, prednisone, prednival, prednylidene, rimexolone, tixocortol, triamcinolone, triamcinolone acetonide, triamcinolone benetonide, and triamcinolone hexacetonide.

In some embodiments, the second therapeutic agent is selected from the group consisting of angiogenesis inhibitors, monoclonal antibodies to adhesion molecules and growth factors, ICE inhibitors, 5-HT3 receptor antagonists e.g. tropisetrone, p38 mitogen activated protein kinase inhibitors, matrix metalloproteinase inhibitors, lymphokine antagonists such as IL-1 receptor antagonists or anti-tumor necrosis factor a agents, e.g. etanercept, or infliximab.

For combination therapies described herein, dosages of the co-administered compounds vary depending on the type of co-drug(s) employed, on the specific drug(s) employed, on the disease or condition being treated and so forth. In additional embodiments, when co-administered with one or more other therapeutic agents, the compound provided herein is administered either simultaneously with the one or more other therapeutic agents, or sequentially.

If administration is simultaneous, the multiple therapeutic agents are, by way of example only, provided in a single, unified form, or in multiple forms.

### Exemplary Embodiments

In addition to the other aspects and embodiments disclosed herein, the following embodiments are specifically contemplated.
1. A method comprising the steps of identifying a patient having an autoimmune disease and administering to said patient a compound of Formula (1).
2. A method comprising the steps of identifying a patient having ANCA associated vasculitis, antibody mediated transplant rejection, autoimmune hemolytic anemia, IgG4-related disease, chronic idiopathic demyelinating polyneuropathy (CIDP), graft-versus-host disease, or neuromyelitis optica and administering to said patient a compound that inhibits the activity of one or more conditions selected from the group consisting of Tfh cells, B-cells, germinal centers, extrafollicular T and B-cells, and ectopic follicular structures.
3. A method comprising the steps of identifying a patient having ANCA associated vasculitis, antibody mediated transplant rejection, autoimmune hemolytic anemia, IgG4-related disease, chronic idiopathic demyelinating polyneuropathy (CIDP), graft-versus-host disease, or neuromyelitis optica and administering to said patient a compound of Formula (I).
4. A method comprising the steps of identifying a patient having ANCA associated vasculitis and administering to said patient a compound of Formula (I).
5. A method comprising the steps of identifying a patient having antibody mediated transplant rejection and administering to said patient a compound of Formula (I).
6. A method comprising the steps of identifying a patient having autoimmune hemolytic anemia and administering to said patient a compound of Formula (I).
7. A method comprising the steps of identifying a patient having IgG4-related disease and administering to said patient a compound of Formula (I).
8. A method comprising the steps of identifying a patient having chronic idiopathic demyelinating polyneuropathy (CIDP) and administering to said patient a compound of Formula (I).
9. A method comprising the steps of identifying a patient having graft-versus-host disease and administering to said patient a compound of Formula (I).
10. A method comprising the steps of identifying a patient having neuromyelitis optica and administering to said patient a compound of Formula (I).
11. A method comprising the steps of identifying a patient as having elevated numbers or activity of plasmablasts in blood or tissue and administering to the patient a compound of Formula (I).
12. A method comprising the steps of identifying a patient as having elevated numbers or activity of plasmablasts in blood or tissue and administering to the patient a compound of Formula (I); wherein said patient has been diagnosed with an autoimmune disease.
13. A method comprising the steps of identifying a patient as having a plasmablast signature as determined by RNA expression or protein expression analyses and administering to the patient a compound of Formula (I).
14. A method comprising the steps of identifying a patient as having a plasmablast signature as determined by RNA expression or protein expression analyses and administering to the patient a compound of Formula (I); wherein said patient has been diagnosed with an autoimmune disease.
15. A method comprising the steps of identifying a patient as having elevated numbers or activity of Tfh cells or Tfh-like cells in blood or tissue and administering to the patient a compound of Formula (I).
16. A method comprising the steps of identifying a patient as having elevated numbers or activity of Tfh cells or Tfh-like cells in blood or tissue and administering to the patient a compound of Formula (I); wherein said patient has been diagnosed with an autoimmune disease.
17. A method comprising the steps of identifying a patient as having elevated numbers or activity of germinal centers or extra follicular T and B-cells in blood and administering to the patient a compound of Formula (I).
18. A method comprising the steps of identifying a patient as having elevated numbers or activity of germinal centers or extra follicular T and B-cells and administering to the patient a compound of Formula (I); wherein said patient has been diagnosed with an autoimmune disease.
19. A method comprising the steps of identifying a patient as having the presence of ectopic follicular structures or ectopic germinal centers or ectopic lymphoid aggregates in a tissue and administering to the patient a compound of Formula (I).
20. A method comprising the steps of identifying a patient as having the presence of ectopic follicular structures or ectopic germinal centers or ectopic lymphoid aggregates in a tissue and administering to the patient a compound of Formula (I); wherein said patient has been diagnosed with an autoimmune disease.
21. A method comprising the steps of identifying a patient as having elevated levels of autoantibodies or immune complexes in blood or tissue and administering to the patient a compound of Formula (I).

A method comprising the steps of identifying a patient as having elevated levels of autoantibodies or immune complexes in blood or tissue and administering to the patient a compound of Formula (I); wherein said patient has been diagnosed with an autoimmune disease.
22. The method of any of the preceding embodiments, wherein said autoimmune disease is one or more conditions selected from the group consisting of lupus erythematosus, ankylosing spondylitis, Chagas disease, chronic idiopathic demyelinating polyneuropathy (CIDP), chronic obstructive pulmonary disease, Crohn's Disease, dermatomyositis, diabetes mellitus type 1, endometriosis, Goodpasture syndrome, Graves' disease, Guillain-Barré syndrome (GBS), Hashimoto's disease, hidradenitis suppurativa, juvenile dermatomyositis, Kawasaki disease, IgA nephropathy, idiopathic thrombocytopenic purpura, IgG4-related disease, interstitial cystitis, mixed connective tissue disease, morphea, multiple sclerosis, secondary progressive multiple sclerosis, myasthenia gravis, narcolepsy, neuromyelitis optica, neuromyotonia, pemphigus vulgaris, pernicious anemia, psoriasis, psoriatic arthritis, polymyositis, primary biliary cirrhosis, relapsing polychondritis, rheumatoid arthritis, sarcoidosis, schizophrenia, scleroderma, Sjögren's syndrome, stiff person syndrome, temporal arteritis, ulcerative colitis, vasculitis, vitiligo, Wegener's granulomatosis, and any combination thereof.
23. The method of any of the preceding embodiments, wherein said autoimmune disease is lupus erythematosus,
24. The method of any of the preceding embodiments, wherein said autoimmune disease is lupus nephritis
25. The method of any of the preceding embodiments, wherein said autoimmune disease is ANCA associated vasculitis.
26. The method of any of the preceding embodiments, wherein said autoimmune disease is ankylosing spondylitis.
27. The method of any of the preceding embodiments, wherein said autoimmune disease is antibody mediated transplant rejection.
28. The method of any of the preceding embodiments, wherein said autoimmune disease is autoimmune hemolytic anemia,
29. The method of any of the preceding embodiments, wherein said autoimmune disease is Chagas disease.
30. The method of any of the preceding embodiments, wherein said autoimmune disease is chronic idiopathic demyelinating polyneuropathy (CIDP).
31. The method of any of the preceding embodiments, wherein said autoimmune disease is chronic obstructive pulmonary disease.
32. The method of any of the preceding embodiments, wherein said autoimmune disease is Crohn's Disease.
33. The method of any of the preceding embodiments, wherein said autoimmune disease is dermatomyositis.
34. The method of any of the preceding embodiments, wherein said autoimmune disease is diabetes mellitus type 1.
35. The method of any of the preceding embodiments, wherein said autoimmune disease is endometriosis.
36. The method of any of the preceding embodiments, wherein said autoimmune disease is Goodpasture syndrome.
37. The method of any of the preceding embodiments, wherein said autoimmune disease is graft-versus-host disease,
38. The method of any of the preceding embodiments, wherein said autoimmune disease is Graves' disease.
39. The method of any of the preceding embodiments, wherein said autoimmune disease is Guillain-Barré syndrome (GBS).
40. The method of any of the preceding embodiments, wherein said autoimmune disease is Hashimoto's disease.
41. The method of any of the preceding embodiments, wherein said autoimmune disease is hidradenitis suppurativa
42. The method of any of the preceding embodiments, wherein said autoimmune disease is juvenile dermatomyositis.
43. The method of any of the preceding embodiments, wherein said autoimmune disease is Kawasaki disease.
44. The method of any of the preceding embodiments, wherein said autoimmune disease is IgA nephropathy.
45. The method of any of the preceding embodiments, wherein said autoimmune disease is idiopathic thrombocytopenic purpura.
46. The method of any of the preceding embodiments, wherein said autoimmune disease is IgG4-related disease.
47. The method of any of the preceding embodiments, wherein said autoimmune disease is interstitial cystitis.
48. The method of any of the preceding embodiments, wherein said autoimmune disease is mixed connective tissue disease.
49. The method of any of the preceding embodiments, wherein said autoimmune disease is morphea.
50. The method of any of the preceding embodiments, wherein said autoimmune disease is multiple sclerosis.
51. The method of any of the preceding embodiments, wherein said autoimmune disease is secondary progressive multiple sclerosis.
52. The method of any of the preceding embodiments, wherein said autoimmune disease is myasthenia gravis.
53. The method of any of the preceding embodiments, wherein said autoimmune disease is narcolepsy.
54. The method of any of the preceding embodiments, wherein said autoimmune disease is neuromyelitis optica.
55. The method of any of the preceding embodiments, wherein said autoimmune disease is neuromyotonia.
56. The method of any of the preceding embodiments, wherein said autoimmune disease is pemphigus vulgaris.
57. The method of any of the preceding embodiments, wherein said autoimmune disease is pernicious anemia.
58. The method of any of the preceding embodiments, wherein said autoimmune disease is psoriasis.
59. The method of any of the preceding embodiments, wherein said autoimmune disease is psoriatic arthritis.
60. The method of any of the preceding embodiments, wherein said autoimmune disease is polymyositis.
61. The method of any of the preceding embodiments, wherein said autoimmune disease is primary biliary cirrhosis.
62. The method of any of the preceding embodiments, wherein said autoimmune disease is relapsing polychondritis.
63. The method of any of the preceding embodiments, wherein said autoimmune disease is rheumatoid arthritis.
64. The method of any of the preceding embodiments, wherein said autoimmune disease is sarcoidosis.
65. The method of any of the preceding embodiments, wherein said autoimmune disease is schizophrenia.
66. The method of any of the preceding embodiments, wherein said autoimmune disease is scleroderma.
67. The method of any of the preceding embodiments, wherein said autoimmune disease is Sjögren's syndrome.
68. The method of any of the preceding embodiments, wherein said autoimmune disease is stiff person syndrome.
69. The method of any of the preceding embodiments, wherein said autoimmune disease is temporal arteritis.
70. The method of any of the preceding embodiments, wherein said autoimmune disease is ulcerative colitis.
71. The method of any of the preceding embodiments, wherein said autoimmune disease is vasculitis.
72. The method of any of the preceding embodiments, wherein said autoimmune disease is vitiligo.
73. The method of any of the preceding embodiments, wherein said autoimmune disease is Wegener's granulomatosis.
74. The method of any of the preceding embodiments, wherein the compound of Formula (I) is is administered to the subject in an amount of about 0.01 mg/kg to about 1000 mg/kg.
75. The method of any of the preceding embodiments, wherein the compound of Formula (I) is administered to the subject orally, parenterally, intranasally, by inhaling, buccally, topically, or transdermally.
76. The method of any of the preceding embodiments, wherein the compound of Formula (I) is administered to the subject orally.
77. The method of any of the preceding embodiments, wherein the compound of Formula (I) is administered to the subject once daily.
78. The method of any of the preceding embodiments, wherein the compound of Formula (I) is administered to the subject once weekly.
79. The method of any of the preceding embodiments, wherein the compound of Formula (I) is administered to the subject for about four, eight, or twelve weeks.
80. The method of any of the preceding embodiments, wherein the compound of Formula (I) has the structure:

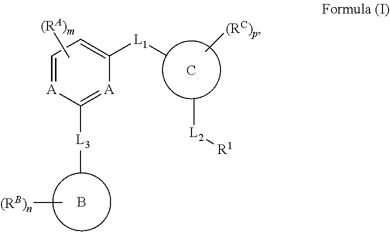

wherein,
each A is independently N or CR;
ring B is heterocycloalkyl or heteroaryl;
ring C is 6-5 fused heterocycle, 6-6 fused heterocycle, or 5-6 fused heterocycle;
L₁ is —(CR²R³)ₓ—, —NR⁴—, —O—, —S—, —O—(CR²R³)ₓ—, —(CR²R³)ₓ—O—, —C(═O)—, —S(═O)—, —S(═O)₂—, —S(═O)₂NR⁴—, —NR⁴S(═O)₂—, —C(═O)O—, —OC(═O)—, —C(═O)NR⁴—, —NR⁴C(═O)—, —OC(═O)NR⁴—, —NR⁴C(═O)O—, or —NR⁴C(═O)NR⁴—;
L₂ and L₃ is independently absent, —O—(CR²R³)ₓ—, —(CR²R³)ₓ—O—, —NR⁴—(CR²R³)ₓ—, —(CR²R³)ₓ—NR⁴—, —C(═O)—, —S(═O)—, —S(═O)₂—, —S(═O)₂NR⁴—, —NR⁴S(═O)₂—, —C(═O)O—, —OC(═O)—, —C(═O)NR⁴—, —NR⁴C(═O)—, —OC(═O)NR⁴—, —NR⁴C(═O)O—, —NR⁴C(═O)NR⁴—, or —NR⁴—;
each R^{A}, R^{B}, R^{C}, R, R¹, R², and R³ is independently H, halogen, —CN, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted phenyl, or substituted or unsubstituted heteroaryl, —OR⁴, —N(R⁴)₂, —CH₂OR⁴, —C(═O)R⁴, —C(═O)OR⁴, —C(═O)N(R⁴)₂, —S(═O)R⁴, —S(═O)₂R⁴, or —NR⁴C(═O)R⁴; each R⁴ is independently H, D, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl;
each x is independently 1, 2, or 3;
m is 0, 1, 2, 3, or 4;
n is 0, 1, 2, 3, or 4; and
p is 0, 1, 2, 3, or 4.
81. The method of any of the preceding embodiments, wherein each A of the compound of Formula (I) is N.
82. The method of any of the preceding embodiments, wherein R^{A} of the compound of Formula (I) is a halogen.
83. The method of any of the preceding embodiments, wherein R^{A} of the compound of Formula (I) is F, Cl, or Br.
84. The method of the preceding embodiments, wherein L₁ of the compound of Formula (I) is is —NR⁴—, —O—, or —C(═O)—.
85. The method of the preceding embodiments, wherein ring C of the compound of Formula (I) is selected from the group consisting of:

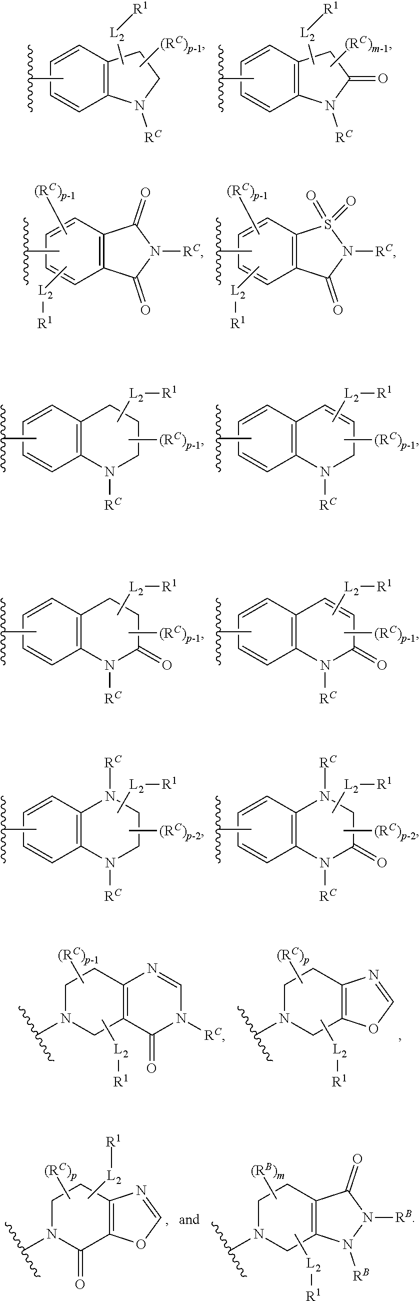

86. The method of any of the preceding embodiments, wherein ring C of the compound of Formula (I) is

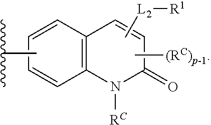

87. The method of any of the preceding embodiments, wherein R^{C} of the compound of Formula (i) is substituted or unsubstituted C₁-C₆alkyl, substituted or unsubstituted C₁-C₆heteroalkyl, or —OR⁴.
88. The method of any of the preceding embodiments, wherein L² of the compound of Formula (I) is —O—(CR²R³)ₓ—.
89. The method of any of the preceding embodiments, wherein R¹ of the compound of Formula (I) is —C(═O)N(R⁴)₂.
90. The method of any of the preceding embodiments, wherein L₃ of the compound of Formula (I) is absent.
91. The method of any of the preceding embodiments, wherein ring B of the compound of Formula (I) is heterocycloalkyl.
92. The method of any of the preceding embodiments, wherein ring B of the compound of Formula (I) is

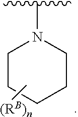

93. The method of any of the preceding embodiments, wherein R^{B} of the compound of Formula (I) is substituted or unsubstituted C₁-C₆alkyl or —C(═O)N(R⁴)₂.
94. The method of any of the preceding embodiments, wherein n of the compound of Formula (I) is 1 or 2.
95. The method of any of the preceding embodiments, wherein ring C of the compound of Formula (I) is

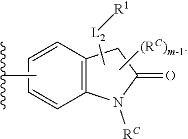

96. The method of any of the preceding embodiments, wherein L₂ of the compound of Formula (I) is absent.
97. The method of any of the preceding embodiments, wherein R¹ and R^{C} of the compound of Formula (I) are H.
98. The method of any of the preceding embodiments, wherein L₃ of the compound of Formula (I) is —NR⁴—(CR²R³)ₓ—.
99. The method of any of the preceding embodiments, wherein ring B of the compound of Formula (I) is heteroaryl.
100. The method of any of the preceding embodiments, wherein ring B of the compound of Formula (I) is

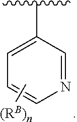

101. The method of any of the preceding embodiments wherein the compound of Formula (I) is 2-((6-((5-chloro-2-((3R,5S)-3,5-dimethylpiperidin-1-yl)pyrimidin-4-yl)amino)-1-methyl-2-oxo-1,2-dihydroquinolin-3-yl)oxy)-N-methylacetamide.
102. The method of any of the preceding embodiments wherein the compound of Formula (I) is 1-(5-chloro-4-((8-methoxy-1-methyl-3-(2-(methylamino)-2-oxoethoxy)-2-oxo-1,2-dihydroquinolin-6-yl)amino)pyrimidin-2-yl)-N,N-dimethylpiperidine-4-carboxamide.
103. The method of any of the preceding embodiments wherein the compound of Formula (I) is 5-((5-chloro-2-((pyridin-3-ylmethyl)amino)pyrimidin-4-yl)amino)indolin-2-one.
   1A. A method of inhibiting Tfh activity in a subject in need thereof, comprising administering to the subject in need thereof a therapeutically effective amount of a BCL6 inhibitor, wherein the BCL6 inhibitor is a compound of Formula (I):

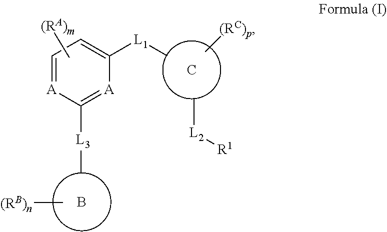

wherein,
each A is independently N or CR;
ring B is heterocycloalkyl or heteroaryl;
ring C is 6-5 fused heterocycle, 6-6 fused heterocycle, or 5-6 fused heterocycle;
L₁ is —(CR²R³)ₓ—, —NR⁴—, —O—, —S—, —O—(CR²R³)ₓ—, —(CR²R³)ₓ—O—, —C(═O)—, —S(═O)—, —S(═O)₂—, —S(═O)₂NR⁴—, —NR⁴S(═O)₂—, —C(═O)O—, —OC(═O)—, —C(═O)NR⁴—, —NR⁴C(═O)—, —OC(═O)NR⁴—, —NR⁴C(═O)O—, or —NR⁴C(═O)NR⁴—;
L₂ and L₃ is independently absent, —O—(CR²R³)ₓ—, —(CR²R³)ₓ—O—, —NR⁴—(CR²R³)ₓ—, —(CR²R³)ₓ—NR⁴—, —C(═O)—, —S(═O)—, —S(═O)₂—, —S(═O)₂NR⁴—, —NR⁴S(═O)₂—, —C(═O)O—, —OC(═O)—, —C(═O)NR⁴—, —NR⁴C(═O)—, —OC(═O)NR⁴—, —NR⁴C(═O)O—, —NR⁴C(═O)NR⁴—, or NR₄—;
each R^{A}, R^{B}, R^{C}, R, R¹, R², and R³ is independently H, halogen, —CN, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted phenyl, or substituted or unsubstituted heteroaryl, —OR⁴, —N(R⁴)₂, —CH₂OR⁴, —C(═O)R⁴, —C(═O)OR⁴, —C(═O)N(R⁴)₂, —S(═O)R⁴, —S(═O)₂R⁴, or —NR⁴C(═O)R⁴;
each R⁴ is independently H, D, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl;
each x is independently 1, 2, or 3;
m is 0, 1, 2, 3, or 4;
n is 0, 1, 2, 3, or 4; and
p is 0, 1, 2, 3, or 4.
   2A. The method of embodiment 1A, wherein each A is N.
   3A. The method of embodiment 1A or 2A, wherein R^{A} is halogen.
   4A. The method of embodiment 3A, wherein R^{A} is F, Cl, or Br.
   5A. The method of any one of embodiments 1A-4A, wherein L₁ is —NR⁴—, —O—, or —C(═O)—.
   6A. The method of any one of embodiments 1A-5A, wherein ring C is selected from the group consisting of:

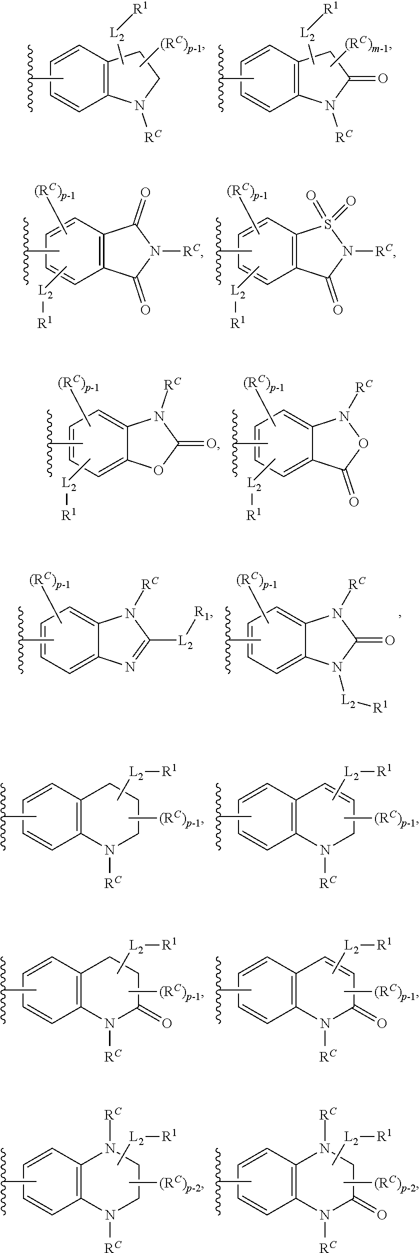

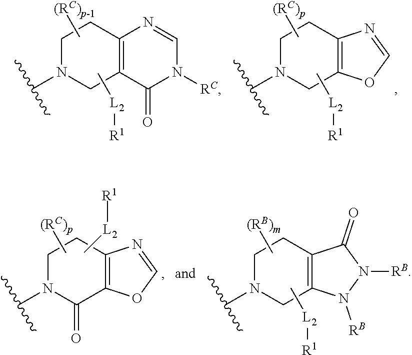

7A. The method of embodiment 6A, wherein ring C is

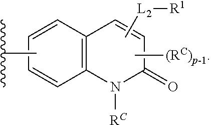

8A. The method of embodiment 7A, wherein R^{C} is substituted or unsubstituted C₁-C₆alkyl, substituted or unsubstituted C₁-C₆heteroalkyl, or —OR⁴.
9A. The method of embodiment 7A, wherein L² is —O—(CR²R³)ₓ—.
10A. The method of embodiment 7A, wherein R¹ is —C(═O)N(R⁴)₂.
11A. The method of embodiment 7A, wherein L₃ is absent.
12A. The method of embodiment 11A, wherein ring B is heterocycloalkyl.
13A. The method of embodiment 12A, wherein ring B is

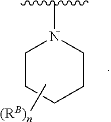

14A. The method of embodiment 13A, wherein R^{B} is substituted or unsubstituted C₁-C₆alkyl or —C(═O)N(R⁴)₂.
15A. The method of embodiment 13A, wherein n is 1 or 2.
16A. The method of embodiment 6A, wherein ring C is

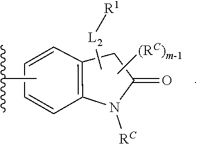

17A. The method of embodiment 16A, wherein L₂ is absent.
18A. The method of embodiment 16A, wherein R¹ and R^{C} are H.
19A. The method of embodiment 16A, wherein L₃ is —NR⁴—(CR²R³)ₓ—.
20A. The method of embodiment 19A, wherein ring B is heteroaryl.
21A. The method of embodiment 20A, wherein ring B is

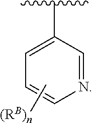

22A. The method of embodiment 1A, wherein the compound of Formula (I) is

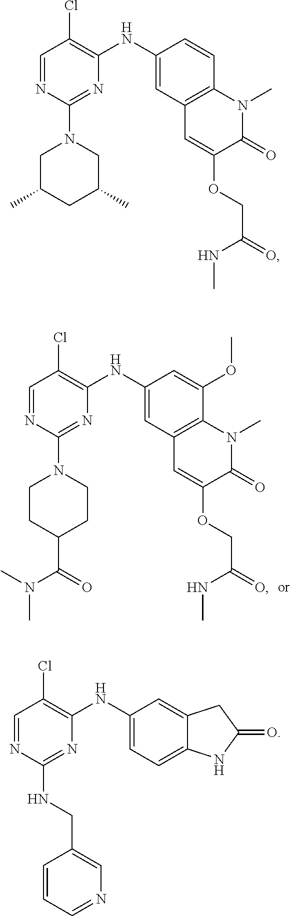 [SMILES: CNC(=O)COc1cc2cc(Nc3nc(N4C[C@H](C)C[C@H](C)C4)ncc3Cl)ccc2n(C)c1=O ; CNC(=O)COc1cc2cc(Nc3nc(N4CCC(C(=O)N(C)C)CC4)ncc3Cl)cc(OC)c2n(C)c1=O ; O=C1Cc2cc(Nc3nc(NCc4cccnc4)ncc3Cl)ccc2N1]

23A. The method of embodiment 1A, wherein the subject suffers from an autoimmune disease.
24A. The method of embodiment 23A, wherein the autoimmune disease is selected from the group consisting of lupus erythematosus, lupus nephritis, ANCA associated vasculitis, ankylosing spondylitis, antibody mediated transplant rejection, autoimmune hemolytic anemia, Chagas disease, chronic idiopathic demyelinating polyneuropathy (CIDP), chronic obstructive pulmonary disease, Crohn's Disease, dermatomyositis, diabetes mellitus type 1, endometriosis, Goodpasture syndrome, graft-versus-host disease, Graves' disease, Guillain-Barré syndrome (GBS), Hashimoto's disease, hidradenitis suppurativa, juvenile dermatomyositis, Kawasaki disease, IgA nephropathy, idiopathic thrombocytopenic purpura, IgG4-related disease, interstitial cystitis, mixed connective tissue disease, morphea, multiple sclerosis, secondary progressive multiple sclerosis, myasthenia gravis, narcolepsy, neuromyelitis optica, neuromyotonia, pemphigus vulgaris, pernicious anemia, psoriasis, psoriatic arthritis, polymyositis, primary biliary cirrhosis, relapsing polychondritis, rheumatoid arthritis, sarcoidosis, schizophrenia, scleroderma, Sjögren's syndrome, stiff person syndrome, temporal arteritis, ulcerative colitis, vasculitis, vitiligo, Wegener's granulomatosis, and any combination thereof.
25A. The method of embodiment 24A, wherein the autoimmune disease is lupus erythematosus.
26A. The method of embodiment 25A, wherein the lupus erythematosus is lupus nephritis.
27A. The method of embodiment 24A, wherein the autoimmune disease is IgG4-related disease.
28A. The method of embodiment 24A, wherein the autoimmune disease is Sjögren's syndrome.
29A. The method of embodiment 24A, wherein the autoimmune disease is rheumatoid arthritis.
30A. The method of embodiment 24A, wherein the autoimmune disease is juvenile dermatomyositis.
31A. The method of embodiment 24A, wherein the autoimmune disease is multiple sclerosis.
32A. The method of embodiment 1A, wherein the BCL6 inhibitor is administered to the subject in an amount of about 0.01 mg/kg to about 1000 mg/kg.
33A. The method of embodiment 1A, wherein the BCL6 inhibitor is administered to the subject orally, parenterally, intranasally, by inhaling, buccally, topically, or transdermally.
34A. The method of embodiment 33A, wherein the BCL6 inhibitor is administered to the subject orally.
35A. The method of embodiment 33A, wherein the BCL6 inhibitor is administered to the subject once daily.
36A. The method of embodiment 33A, wherein the BCL6 inhibitor is administered to the subject once weekly.
37A. A method of treating an autoimmune disease in a subject in need thereof, comprising administering to the subject in need thereof a therapeutically effective amount of a BCL6 inhibitor, wherein the BCL6 inhibitor is a compound of Formula (I):

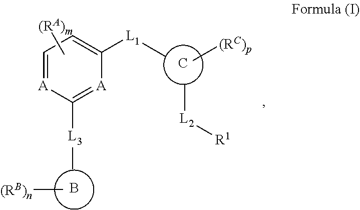

wherein,
each A is independently N or CR;
ring B is heterocycloalkyl or heteroaryl;
ring C is 6-5 fused heterocycle, 6-6 fused heterocycle, or 5-6 fused heterocycle;
L₁ is —(CR²R³)ₓ—, —NR⁴—, —O—, —S—, —O—(CR²R³)ₓ—, —(CR²R³)ₓ—O—, —C(═O)—, —S(═O)—, —S(═O)₂—, —S(═O)₂NR⁴—, —NR⁴S(═O)₂—, —C(═O)O—, —OC(═O)—, —C(═O)NR⁴—, —NR⁴C(═O)—, —OC(═O)NR⁴—, —NR⁴C(═O)O—, or —NR⁴C(═O)NR⁴—;
L₂ and L₃ is independently absent, —O—(CR²R³)ₓ—, —(CR²R³)ₓ—O—, —NR⁴—(CR²R³)ₓ—, —(CR²R³)ₓ—NR⁴—, —C(═O)—, —S(═O)—, —S(═O)₂—, —S(═O)₂NR⁴—, —NR⁴S(═O)₂—, —C(═O)O—, —OC(═O)—, —C(═O)NR⁴—, —NR⁴C(═O)—, —OC(═O)NR⁴—, —NR⁴C(═O)O—, —NR⁴C(═O)NR⁴—, or NR₄—;
each R^{A}, R^{B}, R^{C}, R, R¹, R², and R³ is independently H, halogen, —CN, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted phenyl, or substituted or unsubstituted heteroaryl, —OR⁴, —N(R⁴)₂, —CH₂OR⁴, —C(═O)R⁴, —C(═O)OR⁴, —C(═O)N(R⁴)₂, —S(═O)R⁴, —S(═O)₂R⁴, or —NR⁴C(═O)R⁴;
each R⁴ is independently H, D, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl;
each x is independently 1, 2, or 3;
m is 0, 1, 2, 3, or 4;
n is 0, 1, 2, 3, or 4; and
p is 0, 1, 2, 3, or 4.
   38A. The method of embodiment 37A, wherein each A is N.
   39A. The method of embodiment 37A or 38A, wherein R^{A} is halogen.
   40A. The method of embodiment 39A, wherein R^{A} is F, Cl, or Br.
   41A. The method of any one of embodiments 37A-40A, wherein L₁ is —NR⁴—, —O—, or —C(═O)—.
   42A. The method of any one of embodiments 37A-41A, wherein ring C is selected from the group consisting of:

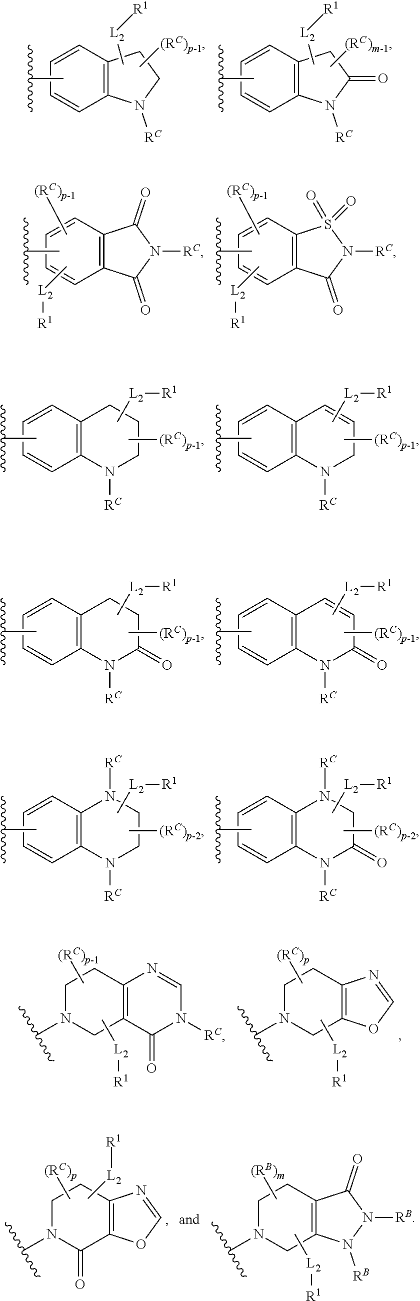

43A. The method of embodiment 42A, wherein ring C is

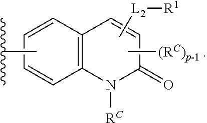

44A. The method of embodiment 43A, wherein R^{C} is substituted or unsubstituted C₁-C₆alkyl, substituted or unsubstituted C₁-C₆heteroalkyl, or —OR⁴.
45 A. The method of embodiment 43A, wherein L² is —O—(CR²R³)ₓ—.
46 A. The method of embodiment 43A, wherein R¹ is —C(═O)N(R⁴)₂.
47 A. The method of embodiment 43A, wherein L₃ is absent.
48 A. The method of embodiment 47A, wherein ring B is heterocycloalkyl.
49 A. The method of embodiment 48A, wherein ring B is

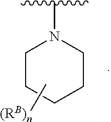

50 A. The method of embodiment 49A, wherein R^{B} is substituted or unsubstituted C₁-C₆alkyl or —C(═O)N(R⁴)₂.
51 A. The method of embodiment 49A, wherein n is 1 or 2.
52 A. The method of embodiment 42A, wherein ring C is

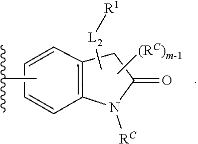

53 A. The method of embodiment 52A, wherein L₂ is absent.
54A. The method of embodiment 52A, wherein R¹ and R^{C} are H.
55 A. The method of embodiment 52A, wherein L₃ is —NR⁴—(CR²R³)ₓ—.
56A. The method of embodiment 55A, wherein ring B is heteroaryl.
57 A. The method of embodiment 56A, wherein ring B is

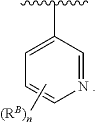

58 A. The method of embodiment 37A, wherein the compound of Formula (I) is

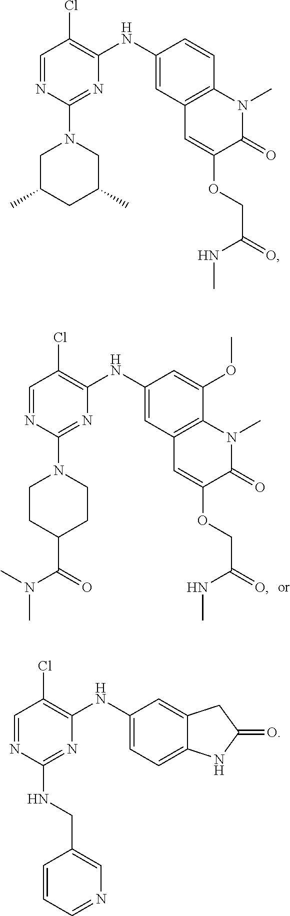 [SMILES: CNC(=O)COc1cc2cc(Nc3nc(N4C[C@H](C)C[C@H](C)C4)ncc3Cl)ccc2n(C)c1=O ; CNC(=O)COc1cc2cc(Nc3nc(N4CCC(C(=O)N(C)C)CC4)ncc3Cl)cc(OC)c2n(C)c1=O ; O=C1Cc2cc(Nc3nc(NCc4cccnc4)ncc3Cl)ccc2N1]

59A. The method of embodiment 37A, wherein the BCL6 inhibitor inhibits non-specific stimulation of B cells.
60 A. The method of embodiment 37A, wherein the BCL6 inhibitor ameliorates autoimmune disease symptoms in the subject.
61 A. The method of embodiment 37A, wherein the BCL6 inhibitor reduces secretion of auto-antibodies by B cells in the subject in vitro.
62 A. The method of embodiment 37A, wherein the BCL6 inhibitor reduces secretion of auto-antibodies by B cells in the subject in vivo.
63 A. The method of embodiment 37A, wherein the method further comprises detecting a first level of one or more auto-antibodies and activated Tfh cells in a first blood sample derived from the subject before administering the BCL6 inhibitor.
64 A. The method of embodiment 63A, wherein the first level of one or more of auto-antibodies and activated Tfh cells in the first blood sample derived from the subject is increased compare to a control before administering the BCL6 inhibitor.
65 A. The method of embodiment 63A, wherein the method further comprises detecting a second level of one or more of auto-antibodies and activated Tfh cells in a second blood sample derived from the subject after administering the BCL6 inhibitor.
66 A. The method of embodiment 65A, wherein the second level of one or more of auto-antibodies and activated Tfh cells in the second blood sample derived from the subject is decreased compare to the first level after administering the BCL6 inhibitor.
67A. The method of any one of embodiments 63A-66A, wherein the auto-antibody comprises IgG and IgM antibodies.
68 A. The method of embodiment 37A, wherein the BCL6 inhibitor inhibits activation, differentiation or re-activation of Tfh cells.
69A. The method of embodiment 37A, wherein the BCL6 inhibitor is administered to the subject in an amount of about 0.01 mg/kg to about 1000 mg/kg.
70 A. The method of embodiment 37A, wherein the BCL6 inhibitor is administered to the subject orally, parenterally, intranasally, by inhaling, buccally, topically, or transdermally.
71 A. The method of embodiment 70A, wherein the BCL6 inhibitor is administered to the subject orally.
72 A. The method of embodiment 70A, wherein the BCL6 inhibitor is administered to the subject once daily.
73A. The method of embodiment 70A, wherein the BCL6 inhibitor is administered to the subject once weekly.
74A. The method of embodiment 70A, wherein the BCL6 inhibitor is administered to the subject for about four, eight, or twelve weeks.
75A. The method of embodiment 37A, wherein the autoimmune disease is selected from the group consisting of lupus erythematosus, lupus nephritis, ANCA associated vasculitis, ankylosing spondylitis, antibody mediated transplant rejection, autoimmune hemolytic anemia, Chagas disease, chronic idiopathic demyelinating polyneuropathy (CIDP), chronic obstructive pulmonary disease, Crohn's Disease, dermatomyositis, diabetes mellitus type 1, endometriosis, Goodpasture syndrome, graft-versus-host disease, Graves' disease, Guillain-Barré syndrome (GBS), Hashimoto's disease, hidradenitis suppurativa, juvenile dermatomyositis, Kawasaki disease, IgA nephropathy, idiopathic thrombocytopenic purpura, IgG4-related disease, interstitial cystitis, mixed connective tissue disease, morphea, multiple sclerosis, secondary progressive multiple sclerosis, myasthenia gravis, narcolepsy, neuromyelitis optica, neuromyotonia, pemphigus vulgaris, pernicious anemia, psoriasis, psoriatic arthritis, polymyositis, primary biliary cirrhosis, relapsing polychondritis, rheumatoid arthritis, sarcoidosis, schizophrenia, scleroderma, Sjögren's syndrome, stiff person syndrome, temporal arteritis, ulcerative colitis, vasculitis, vitiligo, Wegener's granulomatosis, and any combination thereof.
76 A. The method of embodiment 75A, wherein the autoimmune disease is lupus erythematosus.
77 A. The method of embodiment 76A, wherein the lupus erythematosus is lupus nephritis.
78A. The method of embodiment 75A, wherein the autoimmune disease is IgG4-related disease.
79 A. The method of embodiment 75A, wherein the autoimmune disease is Sjögren's syndrome.
80 A. The method of embodiment 75A, wherein the autoimmune disease is rheumatoid arthritis.
81 A. The method of embodiment 75A, wherein the autoimmune disease is juvenile dermatomyositis.
82 A. The method of embodiment 75A, wherein the autoimmune disease is multiple sclerosis.
83 A. The method of any one of the preceding embodiments wherein the compound of Formula (I) is 2-((6-((5-chloro-2-((3R,5S)-3,5-dimethylpiperidin-1-yl)pyrimidin-4-yl)amino)-1-methyl-2-oxo-1,2-dihydroquinolin-3-yl)oxy)-N-methylacetamide.
84 A. The method of any one of the preceding embodiments wherein the compound of Formula (I) is 1-(5-chloro-4-((8-methoxy-1-methyl-3-(2-(methylamino)-2-oxoethoxy)-2-oxo-1,2-dihydroquinolin-6-yl)amino)pyrimidin-2-yl)-N,N-dimethylpiperidine-4-carboxamide.
85 A. The method of any one of the preceding embodiments wherein the compound of Formula (I) is 5-((5-chloro-2-((pyridin-3-ylmethyl)amino)pyrimidin-4-yl)amino)indolin-2-one.

Other embodiments and uses will be apparent to one skilled in the art in light of the present disclosures. The following examples are provided merely as illustrative of various embodiments and shall not be construed to limit the invention in any way.

### Examples

These examples are provided for illustrative purposes only and not to limit the scope of the claims provided herein. The starting materials and reagents used for the synthesis of the compounds described herein may be synthesized or can be obtained from commercial sources, such as, but not limited to, Sigma-Aldrich, Acros Organics, Fluka, and Fischer Scientific.

### Chemical Synthesis

Exemplary compounds are synthesized according to procedures known in the art. Such compounds can be routinely synthesized by a skilled artisan armed with the guidance presented herein and skill in the art. Exemplary compounds can also be purchased from the commercial sources.

### Pharmaceutical Compositions

### Example A-1: Parenteral Composition

To prepare a parenteral pharmaceutical composition suitable for administration by injection, 100 mg of a water-soluble salt of a compound of Formula (I), or pharmaceutically acceptable solvate thereof, is dissolved in 2% HPMC, 1% Tween 80 in DI water, pH 2.2 with MSA, q.s. to at least 20 mg/mL for intravenous (IV) administration. Compound of Formula (1) or pharmaceutically acceptable solvates thereof, is suspended in 0.1% Tween 80, 0.5% Methylcellulose in water for intraperitoneal (IP) or subcute (SC) Injection.

### Example A-2: Oral Composition

To prepare a pharmaceutical composition for oral delivery, 100 mg of a compound of Formula (I), or pharmaceutically acceptable salt or solvate thereof, is mixed with 750 mg of starch. The mixture is incorporated into an oral dosage unit for, such as a hard gelatin capsule, which is suitable for oral administration.

### Biology Examples

### Example 1: Effects of In Vivo Treatment with Test Compounds of Formula (I) on Anti-KLH Antibody Production in Mice

This study will test IgG antibody production and germinal center activity after in vivo treatment of KLH-immunized mice with Compound 1, Compound 2, and the positive control cyclophosphamide (CP).

### SUMMARY

Immunization of C57BL/6 mice with the T cell dependent antigen KLH induced a robust immune response as judged by serum levels of IgG and PNA staining of splenic germinal centers. Treatment with cyclophosphamide (CP; 50 mg/kg, IP, once weekly over 14 days; Group 4) yielded a nominal but non-significant attenuation of this immune response. In contrast, treatment with 100 mg/kg of the BCL6 inhibitor Compound 2 (IP, once daily over 14 days; Group 6) provided highly significant attenuation of the immune response as demonstrated by reduced IgG levels and germinal center activity.

Treatment with 10 or 30 mg/kg of the—Compound 2 (IP, once daily over 14 days; Group 5 and 4 respectively) demonstrated a less significant attenuation of the immune response to KLH with no observable dose response.

### Materials and Methods

Experimental animals: Female C57BL/6 mice were purchased from Envigo (Livermore, Calif.) to provide 8- to 10-week-old animals at the time of immunization. IgG measurements were performed using Innovative Research KLH IgG ELISA kit. Germinal center staining was performed on Dako autostainer using procedures well known in the art.

Study design: Study design and treatments of all groups are shown in Table I.

TABLE 2
Study Design

| | | Day 0 | Days 0-13 | | | | |
|---|---|---|---|---|---|---|---|
| | | Immunization | | Dose | | | |
| Group | n | (IP) | Treatment | (mg/kg, ip) | Freq. | Volume | Purpose |
| 1 | 5 | PBS | Vehicle | — | QD | 5 | Negative |
| | | | | | | mL/kg | control for |
| | | | | | | | immunization |
| 2 | 10 | KLH | Vehicle | — | QD | 5 | Negative |
| | | | | | | mL/kg | control for |
| | | | | | | | treatment |
| 3 | 10 | KLH | CP | 50 | QW | 5 | Positive |
| | | | | | | mL/kg | control for |
| | | | | | | | treatment |
| 4 | 10 | KLH | Compound 1 | 30 | QD | 5 | Test Article |
| | | | | | | mL/kg | |
| 5 | 10 | KLH | Compound 1 | 10 | QD | 5 | Test Article |
| | | | | | | mL/kg | |
| 6 | 10 | KLH | Compound 2 | 100 | QD | 5 | Test Article |
| | | | | | | mL/kg | |

CP, cyclophosphamide. IP, intraperitoneal. KLH, keyhole limpet hemocyanin, n, number of animals per group. QD, once daily. QW, once weekly.

On Day 0, animals of Groups 2-6 (n=10 each) were immunized by intraperitoneal (IP) injection with 0.2 mL (0.5 mg) per mouse of KLH, a dose determined to be optimal for IP administration without adjuvant. Mice from Group 1 (n=5) were sham-immunized by IP injection of 0.2 mL PBS per mouse.

Starting on Day 0 (immediately post-immunization) and continuing through Day 13 (i.e., 14 days total), animals in Group 1, 2, and 4-6 were injected IP once daily (QD) with vehicle, 30 mg/kg Compound 1, 10 mg/kg Compound 1, or 100 mg/kg Compound 2; animals in Group 3 were injected IP once weekly (QW) with 50 mg/kg CP. All doses were administered by IP injection at 5 mL/kg at approximately the same time (±1 h) each day.

On Day 14 (at approximately 24 hours after the final dose administration), all animals were subjected to terminal cardiocentesis and euthanized. Following clotting and processing, the resulting serum samples were flash-frozen and stored at −80° C. pending analysis by enzyme-linked immunosorbent assay (ELISA). The spleen was harvested from each mouse and split into two. One half of each spleen was collected for immunohistochemical (IHC) analysis of germinal center activity using peanut agglutinin (PNA) staining. Single-cell suspensions were collected from the other spleen halves and stored in DMSO cell-freezing medium. These samples were to be used for optional FACS analysis. Remaining tissues were discarded without further evaluation.

Statistical analysis: Descriptive and inferential analyses were performed using the corresponding functions of Excel 2010 (Microsoft, Redmond, Wash.). Inferential analysis consisted of t-test comparisons (two-tailed, non-paired, heteroscedasticity assumed) between groups as indicated. Values of p<0.05 were considered statistically significant.

Results:KLH-specific IgG Levels. ELISA (performed as experimental replicates) was used to determine the levels of immunoglobulin G (IgG) specific for KLH in the terminal serum samples. The resulting data are provided in Table 3.

TABLE 3

| | | Serum IgG Level, Day 14 | |
|---|---|---|---|
| | | (U/mL) | |
| Group | Treatment | Group Mean | p value* |
| 1 | PBS + Vehicle | −10771 | NA |
| 2 | KLH + Vehicle | 223879 | NA |
| 3 | KLH + CP 50 mpk | 161482 | NS |
| 4 | KLH + Compound 130 mpk | 103098 | <0.01 |
| 5 | KLH + Compound 110 mpk | 100997 | <0.01 |
| 6 | KLH + Compound 2 100 mpk | 41503 | <0.001 |

*p value by Excel two-tailed non-paired t-test comparison to Group 2 (KLH).
NS, not significant (p ≥ 0.05).
NA, not applicable.

Immunohistochemical (IHC) Staining with Peanut Agglutinin (PNA) as a Measure of Germinal Center Activity. IHC with PNA staining was used to determine the levels of positive staining in the germinal centers of the spleen. The resulting data are provided in Table 4.

TABLE 4

| | | % PNA-Positive Staining | | | |
|---|---|---|---|---|---|
| | | of Germinal Center | | Staining Intensity | |
| | | Group | | Group | |
| Group | Treatment | Mean | p value* | Mean | p value* |
| 1 | PBS + | 10% | <0.001 | 1.0 | <0.001 |
| | Vehicle | | | | |
| 2 | KLH + | 42% | NA | 2.4 | NA |
| | Vehicle | | | | |
| 3 | KLH + | 29% | NS | 1.3 | <0.01 |
| | CP 50 mpk | | | | |
| 4 | KLH + | 12% | <0.001 | 1.5 | <0.01 |
| | Compound 1 | | | | |
| | 30 mpk | | | | |
| 5 | KLH + | 12% | <0.001 | 1.2 | <0.001 |
| | Compound 1 | | | | |
| | 10 mpk | | | | |
| 6 | KLH + | 7% | <0.001 | 1.1 | <0.001 |
| | Compound 2 | | | | |
| | 100 mpk | | | | |

*p value by two-tailed non-paired t-test comparison to Group 2 (KLH).
NS, not significant (p ≥ 0.05).
NA, not applicable.

### Example 2: Effects of In Vivo Treatment in the Mouse CIA Model

Collagen induced arthritis (CIA) is an experimental model of human rheumatoid arthritis (RA), an autoimmune disease that is characterized by the attack and degradation of articular cartilage. CIA is induced by immunization with type LL collagen C11, the major constituent protein of articular cartilage, in a susceptible mouse strain, DBA/1. Following immunization, animals develop an autoimmune-mediated polyarthritis that shares several clinical, histological, and immunological features with the human autoimmune disease rheumatoid arthritis. Collagen-induced arthritis (CIA) is a valuable model for elucidating pathogenic mechanisms and evaluating the therapeutic effects of novel compounds on rheumatoid arthritis.

DBA/1 mice develop collagen induced arthritis (CIA) after immunization with collagen emulsified in Complete Freund's Adjuvant (CFA), followed by a booster dose of collagen emulsified in Incomplete Freund's Adjuvant (IFA). Either male or female DBA/1 mice may be used. All mice should be 8 to 12 weeks old at immunization. Mice are usually observed for 40 to 60 days.

Mice will be treated intraperitoneally with test compounds of Formula (I) at doses and frequency according to data from pharmacokinetic studies in mice. Treatment starts at immunization and continues for 6 weeks. Mice are assigned to groups in a balanced manner to achieve similar weight at the time of immunization.

Readouts: clinical scores and body weights. At the end of the study, optional readouts include: collection of joints and spleens for histopathology, and collection of immune cells for flow cytometry.

### Example 3: Tfh Cell Phenotypic Assay from PBMC

Input Cells: CD4+CD45R0+ cells from PBMC

Readout Phenotypes: CD4+ CXCR5+bc16+PD-1hi ICOShi

### Materials and Reagents

1. Peripheral blood mononuclear cells (PBMCs) obtained from buffy coat or whole blood.
2. Phosphate buffered saline (PBS) (1×, Gibco).
3. Roswell Park Memorial Institute 1640 medium (RPMI) (Gibco) supplemented with penicillin G/streptomycin (0.5×, Sigma), L-glutamine (2 mM, Sigma), HEPES solution (25 mM, Gibco), Sodium Pyruvate (1 mM, Sigma), non-essential amino acid (1×, Sigma), 50 mkM b-mercaptoethanol (Sigma), and 10% heat-inactivated FCS (ATCC).
4. Millipore Stericup system 1L receiver/PES membrane 0.22 mkm.
5. 15-ml polystyrene tubes (BD Falcon).
6. 50-ml polystyrene tubes (BD Falcon).
7. 10-ml pipettes and pipette aid (BD Falcon).
8. Labeled cryovials.
9. Cold freezing media (FCS supplemented with 10% dimethylsulfoxide, DMSO).
10. Trypan blue.
11. Hemocytometer.
12. MACS buffer (PBS+2% FCS+2 mM EDTA).
13. MS or LS MACS separation column (Miltenyi Biotech).
14. MACs magnetic separator (Miltenyi Biotech).
15. Human CD19 micro beads (Miltenyi Biotech).
16. Human CD45RO microbeads (Miltenyi Biotech)
17. 5-ml polystyrene round bottom flow cytometer tube (BD Falcon).
18. 5-ml polypropylene round bottom culture tube (BD Falcon).
19. Staining buffer 1% BSA in PBS, sterile filtered.
20. 96 well plate polystyrene round bottom culture plate (TPP).
21. 96-well sterile V-bottom plates (Thermo Fisher Scientific)
22. Live/dead fixable aqua dead cell stain kit (Life Technologies)
23. MACS Cell Separation Kits (TBD)
24. Activating beads anti-CD3 (OKT3 or equivalent) anti-ICOS (BioLegend C398.4A)

### Equipment

1. Centrifuge (Sorvall RC4).
2. Water bath.
3. Biosafety cabinet.
4. Microscope.
5. 37° C. 5% CO₂ incubator.
6. Flow Cytometer
7. MACS magnetic separator

### Procedure

A) Isolation of Memory CD4+ T Cells (cTfh) from PBMC
1. Prepare PMBC by Ficoll gradient
2. Count cells using trypan blue exclusion using a hemocytometer.
3. Cryopreserve cells in FCS supplemented with 10% DMSO. Store in liquid nitrogen freezer until use. 1×10⁸-5×10⁷ cell/tube (if desired)

### Thawing Cells (if Needed)

Set the temperature of the centrifuge at 18-20° C.
1. Warm 1× PBS at 37° C. in water bath.
2. Remove vial from liquid nitrogen or −80° C. freezer.
3. Thaw rapidly in a 37° C. water bath.
4. Pipette contents rapidly into a 15-ml tube. If thawing more than 1 vial, use a 50-ml tube.
5. Mix cells with 10-ml warm PBS per vial.
6. Centrifuge the cells at 1300 rpm for 7 minutes at 18-20° C.
7. Discard the supernatant with aspiration without disturbing the pellet.
8. Wash cells with 10-ml warm PBS per vial.
9. Centrifuge the cells at 1300 rpm for 7 minutes at 18-20° C.
10. Discard the supernatant with aspiration without disturbing the pellet.
11. Wash cells with 10-ml warm PBS per vial.
12. Centrifuge the cells at 1300 rpm for 7 minutes at 18-20° C.
13. Discard the supernatant with aspiration without disturbing the pellet.
14. Add complete RPMI supplemented with 10% FCS. Use 3-ml for 15-ml tube, and 10-ml for 50-ml tube.
15. Incubate cells for 15 minutes in 37° C. incubator.
16. Centrifuge the cells at 1300 rpm for 7 minutes at 18-20° C.
17. Re-suspend cells in 10-ml complete RPMI supplemented with 10% FCS. Mix well.
18. Pass the cells through nylon cell filter (70 um) to remove small cell-clumps.
19. Count cells live and dead. Do not use cells preparations that have viability less than 70%.
20. Proceed immediately to MACS purification.

### MACS Purification

1. Purify CD4+CD45RO+ memory T cells using Miltenyi Memory T Cell Isolation Kit

### Culture of T Cells for Activation

1. Culture at 10,000 cells/well in V- or U-bottom plates for TBD days at 37degC
2. Stimulus
   a. Anti-CD3/ICOS beads (ratio TBD, probably 1:1, Miltenyi)
   b. Medium control with no stimulus
3. Test Article or Positive Control in presence of anti-CD3/ICOS stimulus
   a. Time of addition: initiation of culture
   b. Most likely including minimal DMSO

### FACS Cell Staining for Phenotypic Readout

CD4, CXCR5, PD-1, ICOS, [IL-21 (intracellular stain), bc16 (intracellular stain)] Staining Antibodies for Phenotypic Assay; Anti-CD4 FITC (RPA-T4; BD Pharmigen) 1:200; Anti-CXCR5 BV421 (RF8B2; BD pharmigen) 1:200; Anti-ICOSPE-Cy7 (C398.4A; BioLegend) 1:100; Anti-PD-1 eBioscience or BD EH12.2H7 1:100; Anti-bc16 BD K112-91 1:20; Anti-IL-21 BD to be determined.

What is claimed is:

1. A method comprising the steps of identifying a patient having ANCA associated vasculitis, antibody mediated transplant rejection, autoimmune hemolytic anemia, IgG4-related disease, chronic idiopathic demyelinating polyneuropathy (CIDP), graft-versus-host disease, or neuromyelitis optica and administering to said patient a compound of Formula (I).

2. A method comprising the steps of identifying a patient having ANCA associated vasculitis and administering to said patient a compound of Formula (I).

3. A method comprising the steps of identifying a patient having antibody mediated transplant rejection and administering to said patient a compound of Formula (I).

4. A method comprising the steps of identifying a patient having autoimmune hemolytic anemia and administering to said patient a compound of Formula (I).

5. A method comprising the steps of identifying a patient having IgG4-related disease and administering to said patient a compound of Formula (I).

6. A method comprising the steps of identifying a patient having chronic idiopathic demyelinating polyneuropathy (CIDP) and administering to said patient a compound of Formula (I).

7. A method comprising the steps of identifying a patient having graft-versus-host disease and administering to said patient a compound of Formula (I).

8. A method comprising the steps of identifying a patient having neuromyelitis optica and administering to said patient a compound of Formula (I).

9. A method comprising the steps of identifying a patient as having elevated numbers or activity of plasmablasts in blood or tissue and administering to the patient a compound of Formula (I).

10. A method comprising the steps of identifying a patient as having elevated numbers or activity of plasmablasts in blood or tissue and administering to the patient a compound of Formula (I); wherein said patient has been diagnosed with an autoimmune disease.

11. A method comprising the steps of identifying a patient as having a plasmablast signature as determined by RNA expression or protein expression analyses and administering to the patient a compound of Formula (I).

12. A method comprising the steps of identifying a patient as having a plasmablast signature as determined by RNA expression or protein expression analyses and administering to the patient a compound of Formula (I); wherein said patient has been diagnosed with an autoimmune disease.

13. A method comprising the steps of identifying a patient as having elevated numbers or activity of Tfh cells or Tfh-like cells in blood or tissue and administering to the patient a compound of Formula (I).

14. A method comprising the steps of identifying a patient as having elevated numbers or activity of Tfh cells or Tfh-like cells in blood or tissue and administering to the patient a compound of Formula (I); wherein said patient has been diagnosed with an autoimmune disease.

15. A method comprising the steps of identifying a patient as having elevated numbers or activity of germinal centers or extra follicular T and B-cells in blood and administering to the patient a compound of Formula (I).

16. A method comprising the steps of identifying a patient as having elevated numbers or activity of germinal centers or extra follicular T and B-cells and administering to the patient a compound of Formula (I); wherein said patient has been diagnosed with an autoimmune disease.

17. A method comprising the steps of identifying a patient as having the presence of ectopic follicular structures or ectopic germinal centers or ectopic lymphoid aggregates in a tissue and administering to the patient a compound of Formula (I).

18. A method comprising the steps of identifying a patient as having the presence of ectopic follicular structures or ectopic germinal centers or ectopic lymphoid aggregates in a tissue and administering to the patient a compound of Formula (I); wherein said patient has been diagnosed with an autoimmune disease.

19. A method comprising the steps of identifying a patient as having elevated levels of autoantibodies or immune complexes in blood or tissue and administering to the patient a compound of Formula (I).

20. A method comprising the steps of identifying a patient as having elevated levels of autoantibodies or immune complexes in blood or tissue and administering to the patient a compound of Formula (I); wherein said patient has been diagnosed with an autoimmune disease.

21. The method of any of the preceding claims, wherein said autoimmune disease is one or more conditions selected from the group consisting of lupus erythematosus, lupus nephritis, ANCA associated vasculitis, ankylosing spondylitis, antibody mediated transplant rejection, autoimmune hemolytic anemia, Chagas disease, chronic idiopathic demyelinating polyneuropathy (CIDP), chronic obstructive pulmonary disease, Crohn's Disease, dermatomyositis, diabetes mellitus type 1, endometriosis, Goodpasture syndrome, graft-versus-host disease, Graves' disease, Guillain-Barré syndrome (GBS), Hashimoto's disease, hidradenitis suppurativa, juvenile dermatomyositis, Kawasaki disease, IgA nephropathy, idiopathic thrombocytopenic purpura, IgG4-related disease, interstitial cystitis, mixed connective tissue disease, morphea, multiple sclerosis, secondary progressive multiple sclerosis, myasthenia gravis, narcolepsy, neuromyelitis optica, neuromyotonia, pemphigus vulgaris, pernicious anemia, psoriasis, psoriatic arthritis, polymyositis, primary biliary cirrhosis, relapsing polychondritis, rheumatoid arthritis, sarcoidosis, schizophrenia, scleroderma, Sjögren's syndrome, stiff person syndrome, temporal arteritis, ulcerative colitis, vasculitis, vitiligo, Wegener's granulomatosis, and any combination thereof.

22. The method of any of the preceding claims, wherein said autoimmune disease is lupus erythematosus,

23. The method of any of the preceding claims, wherein said autoimmune disease is lupus nephritis

24. The method of any of the preceding claims, wherein said autoimmune disease is ANCA associated vasculitis.

25. The method of any of the preceding claims, wherein said autoimmune disease is ankylosing spondylitis.

26. The method of any of the preceding claims, wherein said autoimmune disease is antibody mediated transplant rejection.

27. The method of any of the preceding claims, wherein said autoimmune disease is autoimmune hemolytic anemia,

28. The method of any of the preceding claims, wherein said autoimmune disease is Chagas disease.

29. The method of any of the preceding claims, wherein said autoimmune disease is chronic idiopathic demyelinating polyneuropathy (CIDP).

30. The method of any of the preceding claims, wherein said autoimmune disease is chronic obstructive pulmonary disease.

31. The method of any of the preceding claims, wherein said autoimmune disease is Crohn's Disease.

32. The method of any of the preceding claims, wherein said autoimmune disease is dermatomyositis.

33. The method of any of the preceding claims, wherein said autoimmune disease is diabetes mellitus type 1.

34. The method of any of the preceding claims, wherein said autoimmune disease is endometriosis.

35. The method of any of the preceding claims, wherein said autoimmune disease is Goodpasture syndrome.

36. The method of any of the preceding claims, wherein said autoimmune disease is graft-versus-host disease,

37. The method of any of the preceding claims, wherein said autoimmune disease is Graves' disease.

38. The method of any of the preceding claims, wherein said autoimmune disease is Guillain-Barre syndrome (GBS).

39. The method of any of the preceding claims, wherein said autoimmune disease is Hashimoto's disease.

40. The method of any of the preceding claims, wherein said autoimmune disease is hidradenitis suppurativa

41. The method of any of the preceding claims, wherein said autoimmune disease is juvenile dermatomyositis.

42. The method of any of the preceding claims, wherein said autoimmune disease is Kawasaki disease.

43. The method of any of the preceding claims, wherein said autoimmune disease is IgA nephropathy.

44. The method of any of the preceding claims, wherein said autoimmune disease is idiopathic thrombocytopenic purpura.

45. The method of any of the preceding claims, wherein said autoimmune disease is IgG4-related disease.

46. The method of any of the preceding claims, wherein said autoimmune disease is interstitial cystitis.

47. The method of any of the preceding claims, wherein said autoimmune disease is mixed connective tissue disease.

48. The method of any of the preceding claims, wherein said autoimmune disease is morphea.

49. The method of any of the preceding claims, wherein said autoimmune disease is multiple sclerosis.

50. The method of any of the preceding claims, wherein said autoimmune disease is secondary progressive multiple sclerosis.

51. The method of any of the preceding claims, wherein said autoimmune disease is myasthenia gravis.

52. The method of any of the preceding claims, wherein said autoimmune disease is narcolepsy.

53. The method of any of the preceding claims, wherein said autoimmune disease is neuromyelitis optica.

54. The method of any of the preceding claims, wherein said autoimmune disease is neuromyotonia.

55. The method of any of the preceding claims, wherein said autoimmune disease is pemphigus vulgaris.

56. The method of any of the preceding claims, wherein said autoimmune disease is pernicious anemia.

57. The method of any of the preceding claims, wherein said autoimmune disease is psoriasis.

58. The method of any of the preceding claims, wherein said autoimmune disease is psoriatic arthritis.

59. The method of any of the preceding claims, wherein said autoimmune disease is polymyositis.

60. The method of any of the preceding claims, wherein said autoimmune disease is primary biliary cirrhosis.

61. The method of any of the preceding claims, wherein said autoimmune disease is relapsing polychondritis.

62. The method of any of the preceding claims, wherein said autoimmune disease is rheumatoid arthritis.

63. The method of any of the preceding claims, wherein said autoimmune disease is sarcoidosis.

64. The method of any of the preceding claims, wherein said autoimmune disease is schizophrenia.

65. The method of any of the preceding claims, wherein said autoimmune disease is scleroderma.

66. The method of any of the preceding claims, wherein said autoimmune disease is Sjögren's syndrome.

67. The method of any of the preceding claims, wherein said autoimmune disease is stiff person syndrome.

68. The method of any of the preceding claims, wherein said autoimmune disease is temporal arteritis.

69. The method of any of the preceding claims, wherein said autoimmune disease is ulcerative colitis.

70. The method of any of the preceding claims, wherein said autoimmune disease is vasculitis.

71. The method of any of the preceding claims, wherein said autoimmune disease is vitiligo.

72. The method of any of the preceding claims, wherein said autoimmune disease is Wegener's granulomatosis.

73. The method of any of the preceding claims, wherein the compound of Formula (I) is is administered to the subject in an amount of about 0.01 mg/kg to about 1000 mg/kg.

74. The method of any of the preceding claims, wherein the compound of Formula (I) is administered to the subject orally, parenterally, intranasally, by inhaling, buccally, topically, or transdermally.

75. The method of any of the preceding claims, wherein the compound of Formula (I) is administered to the subject orally.

76. The method of any of the preceding claims, wherein the compound of Formula (I) is administered to the subject once daily.

77. The method of any of the preceding claims, wherein the compound of Formula (I) is administered to the subject once weekly.

78. The method of any of the preceding claims, wherein the compound of Formula (I) is administered to the subject for about four, eight, or twelve weeks.

79. The method of any of the preceding claims, wherein the compound of Formula (I) has the structure: 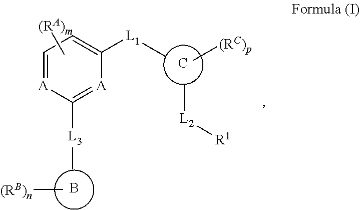
wherein,
each A is independently N or CR;
ring B is heterocycloalkyl or heteroaryl;
ring C is 6-5 fused heterocycle, 6-6 fused heterocycle, or 5-6 fused heterocycle;
L₁ is —(CR²R³)ₓ—, —NR⁴—, —O—, —S—, —O—(CR²R³)ₓ—, —(CR²R³)ₓ—O—, —C(═O)—, —S(═O)—, —S(═O)₂—, —S(═O)₂NR⁴—, —NR⁴S(═O)₂—, —C(═O)O—, —OC(═O)—, —C(═O)NR⁴—, —NR⁴C(═O)—, —OC(═O)NR⁴—, —NR⁴C(═O)O—, or —NR⁴C(═O)NR⁴—;
L₂ and L₃ is independently absent, —O—(CR²R³)ₓ—, —(CR²R³)ₓ—O—, —NR⁴—(CR²R³)ₓ—, —(CR²R³)ₓ—NR⁴—, —C(═O)—, —S(═O)—, —S(═O)₂—, —S(═O)₂NR⁴—, —NR⁴S(═O)₂—, —C(═O)O—, —OC(═O)—, —C(═O)NR⁴—, —NR⁴C(═O)—, —OC(═O)NR⁴—, —NR⁴C(═O)O—, —NR⁴C(═O)NR⁴—, or —NR⁴—;
each R^{A}, R^{B}, R^{C}, R, R¹, R², and R³ is independently H, halogen, —CN, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted phenyl, or substituted or unsubstituted heteroaryl, —OR⁴, —N(R⁴)₂, —CH₂OR⁴, —C(═O)R⁴, —C(═O)OR⁴, —C(═O)N(R⁴)₂, —S(═O)R⁴, —S(═O)₂R⁴, or —NR⁴C(═O)R⁴;
each R⁴ is independently H, D, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl;
each x is independently 1, 2, or 3;
m is 0, 1, 2, 3, or 4;
n is 0, 1, 2, 3, or 4; and
p is 0, 1, 2, 3, or 4.

80. The method of any of the preceding claims, wherein each A of the compound of Formula (I) is N.

81. The method of any of the preceding claims, wherein R^{A} of the compound of Formula (I) is a halogen.

82. The method of any of the preceding claims, wherein R^{A} of the compound of Formula (I) is F, Cl, or Br.

83. The method of the preceding claims, wherein L₁ of the compound of Formula (I) is is —NR⁴—, —O—, or —C(═O)—.

84. The method of the preceding claims, wherein ring C of the compound of Formula (I) is selected from the group consisting of:

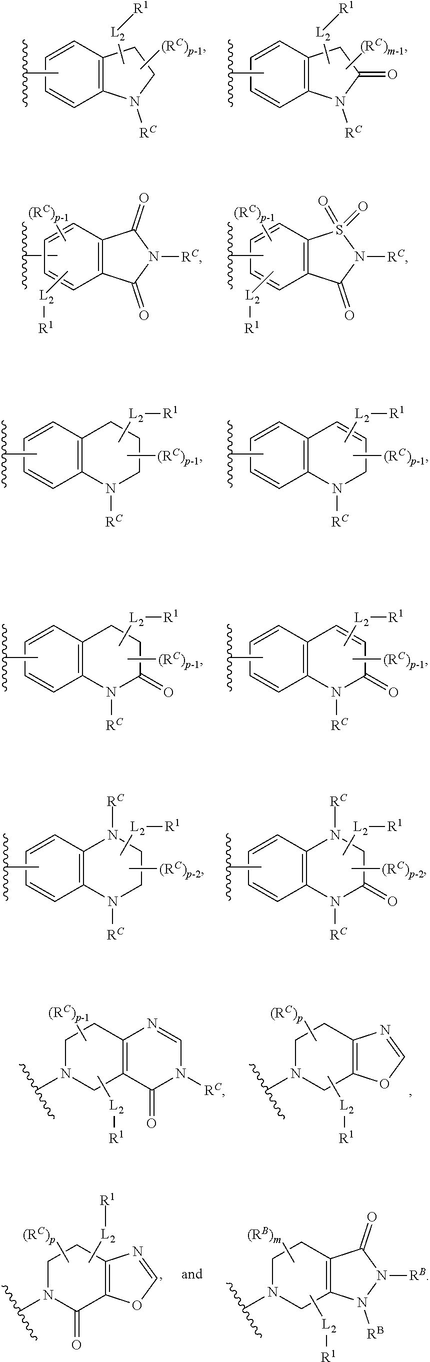

85. The method of any of the preceding claims, wherein ring C of the compound of Formula (I) is

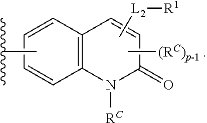

86. The method of any of the preceding claims, wherein R^{C} of the compound of Formula (i) is substituted or unsubstituted C₁-C₆alkyl, substituted or unsubstituted C₁-C₆heteroalkyl, or —OR⁴.

87. The method of any of the preceding claims, wherein L² of the compound of Formula (I) is —O—(CR²R³)ₓ—.

88. The method of any of the preceding claims, wherein R¹ of the compound of Formula (I) is —C(═O)N(R⁴)₂.

89. The method of any of the preceding claims, wherein L₃ of the compound of Formula (I) is absent.

90. The method of any of the preceding claims, wherein ring B of the compound of Formula (I) is heterocycloalkyl.

91. The method of any of the preceding claims, wherein ring B of the compound of Formula (I) is

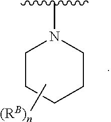

92. The method of any of the preceding claims, wherein R^{B} of the compound of Formula (I) is substituted or unsubstituted C₁-C₆alkyl or —C(═O)N(R⁴)₂.

93. The method of any of the preceding claims, wherein n of the compound of Formula (I) is 1 or 2.

94. The method of any of the preceding claims, wherein ring C of the compound of Formula (I) is

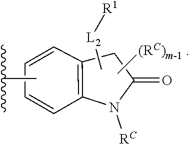

95. The method of any of the preceding claims, wherein L₂ of the compound of Formula (I) is absent.

96. The method of any of the preceding claims, wherein R¹ and R^{C} of the compound of Formula (I) are H.

97. The method of any of the preceding claims, wherein L₃ of the compound of Formula (I) is —NR⁴—(CR²R³)ₓ—.

98. The method of any of the preceding claims, wherein ring B of the compound of Formula (I) is heteroaryl.

99. The method of any of the preceding claims, wherein ring B of the compound of Formula (I) is

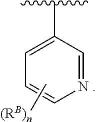

100. The method of any of the preceding claims wherein the compound of Formula (I) is 2-((6-((5-chloro-2-((3R,5S)-3,5-dimethylpiperidin-1-yl)pyrimidin-4-yl)amino)-1-methyl-2-oxo-1,2-dihydroquinolin-3-yl)oxy)-N-methylacetamide.

101. The method of any of the preceding claims wherein the compound of Formula (I) is 1-(5-chloro-4-((8-methoxy-1-methyl-3-(2-(methylamino)-2-oxoethoxy)-2-oxo-1,2-dihydroquinolin-6-yl)amino)pyrimidin-2-yl)-N,N-dimethylpiperidine-4-carboxamide.

102. The method of any of the preceding claims wherein the compound of Formula (I) is 5-((5-chloro-2-((pyridin-3-ylmethyl)amino)pyrimidin-4-yl)amino)indolin-2-one.
